SEQUENCE LISTING (5 sequences)

SEQ ID NO: 1 - DNA, 20 bp, synthetic construct
  source 1..20: mol_type = other DNA
  misc_feature 20: note = 3'-amine
tagctaggaagagtcacaca

SEQ ID NO: 2 - DNA, 20 bp, synthetic construct
  source 1..20: mol_type = other DNA
  misc_feature 20: note = 3'-thiol
tagctaggaagagtcacaca

SEQ ID NO: 3 - DNA, 25 bp, synthetic construct
  source 1..25: mol_type = other DNA
  misc_feature 1: note = 5'-methylene blue
ttttttgtgtgactcttcctagcta

SEQ ID NO: 4 - DNA, 25 bp, synthetic construct
  source 1..25: mol_type = other DNA
  misc_feature 1: note = 5'-methylene blue
ttttttttttttttttttttttttt

SEQ ID NO: 5 - DNA, 92 bp, synthetic construct
  source 1..92: mol_type = other DNA
  misc_feature 1..78: note = monomeric aptamer for SARS-CoV-2 (MSA52)
  misc_feature 92: note = trebler linker attached to TTTTT-3'-thiol
ttacgtcaaggtgtcactcctagggtttggctccgggccggcgtcggtcgtctctcgcgaagcatctctttggcgtgttttttttttttttt

(19) United States
(12) Patent Application Publication
(10) Pub. No.: US 2026/0035576 A1
(43) Pub. Date: Feb. 5, 2026

(54) ANTI-FOULING COATINGS FOR ELECTROCHEMICAL BIOSENSORS

(71) Applicant: McMaster University, Hamilton (CA)

(72) Inventors: Leyla Soleymani, Oakville (CA); Todd Hoare, Ancaster (CA); Survanshu Saxena, Hamilton (CA)

(21) Appl. No.: 19/229,896

(22) Filed: Jun. 5, 2025

Related U.S. Application Data
(60) Provisional application No. 63/656,407, filed on Jun. 5, 2024.

(51) Int. Cl. C09D 5/16; C08F 8/34; C09D 133/14; G01N 27/28; G01N 33/543; G01N 33/68

(52) U.S. Cl. CPC C09D 5/1668; C08F 8/34; C09D 133/14; G01N 27/28; G01N 33/5438; G01N 33/6803

(57) ABSTRACT

Described herein is a zwitterionic polymer-based coating that, when applied to an electrochemical biosensor, is capable of reducing fouling without compromising the current signal while also facilitating probe attachment.

### CROSS-REFERENCE TO RELATED APPLICATION

The present invention claims priority and the benefit of U.S. Provisional Application No. 63/656,407, filed Jun. 5, 2024, the content of which is hereby incorporated herein by reference in its entirety.

### STATEMENT REGARDING ELECTRONIC FILING OF A SEQUENCE LISTING

A Sequence Listing XML file, submitted in accordance with the requirements of 37 C.F.R. § 1.831-835, entitled 180807-00012_ST26.xml, 7,909 bytes in size, generated on Aug. 7, 2025, and filed electronically, is provided in lieu of a paper copy. This Sequence Listing is hereby incorporated herein by reference into the specification for its disclosures.

### FIELD

The present invention relates to polymers, and in particular, polymer coatings for electrodes, such as electrochemical biosensors, to reduce fouling while maintaining high sensing capacity.

### BACKGROUND

The rapid, accurate, and sensitive detection of various disease biomarkers is essential to diagnose and prevent the spread of diseases worldwide, particularly in lower resource settings in which large-scale central testing facilities are less available. Electrochemical biosensors in which a biorecognition element (typically an antibody or DNA) is attached to an electrode surface to enable reporting of binding by changes in resistance and/or current flow are one of the best candidates for such applications due to their high sensitivity, good specificity, low sample volume requirement, rapid detection time, low cost, ability to miniaturize, and easy-to-use format that does not require complex analytical equipment [1-4]. However, given the sensitivity of the resulting signal to any type of binding to the electrode surface, performing biosensing directly in complex biological media such as blood, urine, or saliva can lead to passivation of the biosensing surface based on interactions between various foulants (e.g. proteins, lipids, or cells) and either the biorecognition element or the electrode surface via a variety of non-specific mechanisms (e.g. hydrophobic interactions, electrostatics, or hydrogen bonding interactions) [1,3,5-7]. This fouling process leads to a variety of analytical challenges including an increase in background signal, low signal-to-noise ratios (SNR), a higher number of false positives, lower sensor stability, reduced sample-to-sample reproducibility, and/or compromised sensitivity [1,3,5-9]. While pre-processing of the sample via dilution, filtration, precipitation, centrifugation, or combinations thereof can help reduce fouling in such samples [10,11], pre-processing can lead to reductions in biosensor sensitivity; this is especially true with dilution given that it causes a simultaneous decrease in the target analyte concentration coupled with an increase in the complexity of biosensor use.

As an alternative to sample pre-processing, the use of protein-repellent coatings that do not electrically passivate the surface of the biosensors has attracted significant interest. The majority of electrochemical biosensors use alkanethiol-based small molecule alcohols such as 6-mercapto-1-hexanol (MCH), 2-mercaptocthanol (MCE), or 11-mercaptoundecanoic acid (MUA) as a backfiller for reducing electrode fouling, taking advantage of the strong interaction between gold and thiol groups; correspondingly, thiolated DNA is often used as the biorecognition element. However, major challenges including non-specific background signals due to improper backfilling, poor stability, and low reproducibility have been associated with this approach [5]. The incorporation of thioaromatic self-assembled monolayers (SAMs) such as p-aminothiophenol or p-mercaptobenzoic acid together with alkanethiol-based backfillers or tetrahedral DNA nanostructures have been used to better control the spacing between biorecognition elements present on the biosensor surface and thus improve performance [12-15]; however, the multi-component complexity and thus reproducibility of such coatings represents a drawback of this approach.

Alternately, anti-fouling polymer coatings have been reported for promoting anti-fouling in electrochemical biosensors. Co-assembly of bipodal aromatic poly(ethylene glycol) (PEG)-based alkanethiols and aptamers has been used by Henry et al. to detect genetic markers of breast cancer [16], with the improved anti-fouling effect of PEG offset by the electrically insulative nature of PEG reducing the achievable current. Alternately, combinations of anti-fouling polymers and insoluble conductive polymers have been explored to try to achieve anti-fouling properties without compromising surface conductivity. For example, Shin et al. used a PEG hydrogel combined with —COOH-functionalized poly(3,4-ethylenedioxythiophene) (PEDOT) coated on gold and indium tin oxide (ITO) electrodes for the detection of bovine-interferon-γ in blood [17], Hui et al. used PEGylated polyaniline (PANI) nanofibers coated on glassy carbon electrodes (GCE) to detect the breast cancer susceptibility gene BRCA1 in human serum [18], and Ma et al. developed a hyperbranched polyglycerol (HPG) functionalized with PEDOT for the detection of α-fetoprotein in human serum [19]. However, the high hydrophobicity of the electroactive polymers coupled with the challenging synthesis of these copolymers/graft copolymers increases fabrication costs while offering only moderate anti-fouling benefits.

Various anti-fouling peptides have also been used to impart anti-fouling properties to electrode surfaces and attach biorecognition elements. Carboxylated zwitterionic peptides (EKEKEKE) coupled with PEDOT polymer on GCE have been reported as a conducting and anti-fouling biosensor for detecting BRCA1 [20], while a mixed SAM comprised of aptamers and zwitterionic peptides such as EKEKEKE-PPPPC and EESKSESKSGGGGC on a gold electrode enabled anti-fouling and electrochemical detection of immunoglobulin E (IgE) and α-fetoprotein, respectively [21,22]. Combinations of PEG-based coatings with peptide-based coatings have also been reported, with Gonzalez-Fernandez et al. finding that the inclusion of a six-unit PEG spacer in a peptide-coated electrochemical biosensor offers the best anti-fouling properties compared to spacers with different lengths [23]. However, the use of anti-fouling peptides adds significant cost to the biosensor and requires multi-step synthesis procedures that may be less amenable to scale-up.

Zwitterionic polymers, polymers that contain both cationic and anionic charges in close proximity, have gained increasing interest as alternative coating polymers for both electrodes and more generally biomaterials. The extremely high water binding capacity of zwitterionic functional groups (often enabling >10 water molecules to bind per zwitterionic functionality [24,25]) is key to this interest, enabling better anti-fouling properties than achievable with PEG [26-28]. Multiple types of zwitterionic SAMs have been reported for this purpose. For example, Goda et al. reported the use of a thiolated 2-methacryloyloxyethyl phosphorylcholine (MPC) SAM as an anti-fouling coating on a gold-based biosensor [29], Bertok et al. synthesized the disulfide-bearing sulfobetaine derivative ((R)-3-((2-(5-(1,2-dithiolan-3-yl)-pentanamido)ethyl)dimethylammonio) propane-1-sulfonate (DPS) to fabricate a self-assembled monolayer with MUA on a gold electrode (with a similar coating also used by Jolly et al. for prostate specific antigen (PSA) detection) [30,31], and Wang et al. reported a mixture of thiolated sulfobetaine (SB-thiol) and carboxybetaine (CB-thiol) to form a SAM on gold electrodes [32]. Zwitterionic phenyl layers such as phenyl phosphorylcholine diazonium salts were also reported by Gui et al. to have impedance values lower than PEG alkanethiol-based monolayers [33], while mixed phenyl phosphorylcholine (PPC) and phenyl butyric acid (PBA) SAMs were reported to detect tumor necrosis factor (TNF-α) in whole blood [34]. However, despite the high charge density of zwitterionic polymers, the signal suppression due to the insulative effect of polymers generally has not yet been resolved using these SAM-based approaches. For example, while the CB-thiol monolayers performed better than SB-thiol monolayers in the work of Wang et al., the current in either case was still significantly lower than that achieved with a bare gold surface [32]. Combinations of zwitterionic polymers with pro-adhesive or electrically conductive polymers have also been reported to try to resolve this issue, including polydopaminc/poly(sulfobetaine methacrylate) (PDA-pSBMA) films and poly(sulfobetaine-3,4-ethylenedioxythiophene) (PSBEDOT) films [36]. However, each of these strategies requires multiple components and separate chemical entities for anti-fouling, adhesion, grafting, and/or electrochemical detection, increasing the complexity of fabricating the coating.

### SUMMARY

According to an aspect, provided herein is a polymer coating for an electrochemical biosensor comprising one or more polymerizable zwitterionic monomers containing at least one cationic charge and at least one anionic charge at neutral pH and one or more first polymerizable comonomers that contains or can be modified to contain thiol groups.

In some embodiments, the coating does not require any blocker, spacer, or backfiller to reduce electrode fouling.

In some embodiments, the thiol groups enable covalent, physical, or affinity binding of a sensing probe.

In some embodiments, the first polymerizable comonomer contains or can be modified to contain carboxyl groups.

In some embodiments, the polymer coating further comprises one or more second polymerizable comonomers that enables covalent, physical, or affinity binding of a sensing probe.

In some embodiments, the second polymerizable comonomer contains or can be modified to contain aldehyde groups.

In some embodiments, the zwitterionic monomer comprises a sulfobetaine, carboxybetaine, a phosphorylcholine, or a combination thereof.

In some embodiments, the zwitterionic monomer is [2-(methacryloyloxy)ethyl]dimethyl-(3-sulfopropyl)ammonium hydroxide (DMAPS). In some embodiments, the zwitterionic monomer comprises [2-(methacryloyloxy)ethyl]dimethyl-(3-sulfopropyl)ammonium hydroxide (DMAPS), 2-methacryloyloxyethyl phosphorylcholine (MPC), carboxybetaine methacrylate (CBMA), or combinations thereof.

In some embodiments, the first comonomer is selected from methacrylic acid, acrylic acid, vinylacetic acid, fumaric acid or maleic acid.

In some embodiments, the second comonomer is N-(2,2-dimethoxyethyl) methacrylamide (DMEMA).

In some embodiments, the coating is attached to a noble metal or noble metal-coated electrode via noble metal-thiol interactions. In some embodiments, the noble metal comprises gold. In some embodiments, the coating is attached to a gold or gold-coated electrode via gold-thiol interactions.

In some embodiments, the coating is less than about 20 nm in thickness.

In some embodiments, the coating comprises a sensing probe for detecting a target. In some embodiments, the sensing probe is functionalized with thiol and/or amine groups for binding to the coating. In some embodiments, the coating is functionalized with a sensing probe functionalized with thiol and/or amine groups.

In some embodiments, the sensing probe detects a target.

In some embodiments, the sensing probe comprises a biomolecule, synthetic affinity agent, or combinations thereof.

In some embodiments, the sensing probe is multimeric.

In some embodiments, the biomolecule comprises a nucleic acid, an aptamer, and/or a protein. In some embodiments, the biomolecule comprises a nucleic acid.

In some embodiments, the biomolecule is an aptamer.

In some embodiments, the biomolecule is a protein.

In some embodiments, the target comprises a nucleic acid, a virus, a protein, or a combination thereof. In some embodiments, the target is DNA.

In some embodiments, the target is a virus.

In another aspect, provided herein is an electrochemical biosensor comprising the polymer coating disclosed herein and a device comprising said electrochemical biosensor.

In some embodiments, provided herein is an assay comprising the electrochemical biosensor described herein in combination with magnetic beads labelled to sandwich a target between the polymer coating and the magnetic beads.

Other features and advantages of the present invention will become apparent from the following detailed description. It should be understood, however, that the detailed description and the specific examples, while indicating embodiments of the invention, are given by way of illustration only and the scope of the claims should not be limited by these embodiments but should be given the broadest interpretation consistent with the description as a whole.

### DRAWINGS

Certain embodiments will now be described in greater detail with reference to the attached drawings.

FIG. 1 shows a schematic of the polymerization method used to synthesize the anti-fouling polymers, (a) DMAPS-Ald-MAA-SH polymers and (b) DMAPS-MAA-SH polymers, in exemplary embodiments.

FIG. 2 shows ¹H NMR spectra of (a) DMAPS75-Ald15-MAA-SH10 polymer, (b) DMAPS80-Ald15-MAA-SH5 polymer, (c) DMAPS90-MAA-SH10 polymer, and (d) DMAPS80-MAA-SH20 polymer with the peaks at ˜9.5 ppm indicating the presence of the aldehyde group in the aldehyde-functionalized polymers in exemplary embodiments.

FIG. 3 shows GPC analysis of all the synthesized polymers prior to acid hydrolysis and thiolation in exemplary embodiments.

FIG. 4 shows raw conductometric titration curves of (a) DMAPS75-Ald15-MAA-SH10 polymer, (b) DMAPS80-Ald15-MAA-SH5 polymer, (c) DMAPS90-MAA-SH10 polymer, and (d) DMAPS80-MAA-SH20 polymer before and after thiol modification in exemplary embodiments.

FIG. 5 shows hydroxylamine titration of DMAPS75-Ald15-MAA10 and DMAPS80-Ald15-MAA5 polymer before and after thiol modification-the arrow indicates the pH of the control polymer solution (DMAPS90-MAA10 for polymer without thiol and DMAPS90-MAA-SH-10 for polymer with thiol) that was used as the pH end point for the corresponding experimental samples-in exemplary embodiments.

FIG. 6 shows the geometry of the sputtered gold electrode used for polymer-coating in an exemplary embodiment.

FIG. 7 shows characterization of zwitterionic polymer coatings on a gold surface in exemplary embodiments: (a) fabrication of polymer-coated gold electrodes; (b) HSA-¹²⁵I protein-spiked human plasma adsorption on zwitterionic polymer-coated electrodes compared to bare gold electrodes following one hour of incubation (n=3); (c) anodic current obtained through CV scanning as a function of 1% HSA incubation time on the bare gold and zwitterionic polymer-coated electrodes (n=3); (d) contact angles following coating with different zwitterionic polymer concentrations on a gold surface (n=3); (e) film thickness of the 4 mg mL⁻¹ zwitterionic polymer coating on a gold surface by ellipsometry; (f) confirmation of the presence of free aldehydes on the gold surface by incubating FTSC dye with the zwitterionic polymer-coated surface.

FIG. 8 shows optimization of polymer concentration and composition for effective anti-fouling in exemplary embodiments: (a) protein adsorption studies on gold surfaces coated with polymers prepared with different combinations and mol % of comonomers compared to a bare gold surface (n=3); (b) anodic current obtained through CV scans from the gold surface coated with polymers prepared with different combinations and mol % of comonomers after incubation in 1% HSA over a 24 hour period (n=3); (c) optimization of the concentration of the dip-coating solution of the best-performing anti-fouling polymer from (a) and (b) (DMAPS-75-Ald15-MAA-SH10) according to the optimization of the anodic current obtained from CV scans (n=3).

FIG. 9 shows electrochemical characterization of zwitterionic polymer-coated electrodes using cyclic voltammetry in exemplary embodiments: (a) CV of zwitterionic polymer-coated gold electrodes at different scan rates (10-200 mV s⁻¹), in which (i) shows a plot of the anodic and cathodic current as a function of the square root of the scan rate (n=3) and (ii) shows the difference in peak potential in relation to the square root of scan rate (n=3); (b) CV comparison of zwitterionic polymer-coated and bare gold electrodes at a 50 mV s⁻¹ scan rate (inset shows the mean and standard deviation of the anodic currents obtained in each case) (n=3); ** p≤0.01 obtained from a two-tailed Student's t-test.

FIG. 10 shows electrochemical response of probe-grafted zwitterionic polymer-coated electrochemical sensors in exemplary embodiments: (a) schematics for fabrication of probe-functionalized zwitterionic polymer-coated electrodes; (b-c) CV (b) and EIS (c) scans of the bare gold electrode before and after coating with zwitterionic polymer and grafted with 5+1 μM of thiolated+aminated capture DNA; inset of (c) shows the corresponding Randles circuit; (d) SWV scan of the capture DNA immobilized zwitterionic polymer-coated gold electrodes incubated with 1 μM of complementary reporter DNA for 1 hour at 37° C.; (e) comparison of current obtained from SWV scans upon incubation of buffer (blank), three 1 μM non-complementary reporter DNA sequences (NC-1, NC-2, NC-3) on capture DNA-immobilized zwitterionic polymer-coated gold electrodes, and 1 μM complementary reporter DNA (C-DNA) on capture DNA-immobilized zwitterionic polymer-coated gold electrodes showing biosensor specificity (n=3); Non-significant (p>0.05)=ns, **** p≤0.0001 obtained from two-tailed Student's t-test.

FIG. 11 shows optimization of electrochemical characterization of probe-conjugated zwitterionic polymer-coated electrodes in exemplary embodiments: (a) frequency optimization based on the current obtained from SWV scans at different frequencies (10-800 Hz) (n=3); (b) capture DNA probe type and concentration optimization on polymer-coated electrodes (n=3) indicating that the 5 mM thiol/l mM aminated capture DNA combination results in the highest current signal; (c) capture DNA probe optimization on bare gold electrodes prepared without polymer (n=3) showing saturation of the current at much lower total capture DNA concentrations than observed with the polymer-coated electrodes.

FIG. 12 shows CV curves of a bare gold electrode compared to the same electrode after incubation with 1 μM thiolated capture DNA and backfilled with 100 mM MCH in an exemplary embodiment.

FIG. 13 shows the effects of exploiting the aldehyde-amine interaction between aldehyde-containing polymer coatings and aminated probes for enhancing electrochemical DNA probe detection in exemplary embodiments: (a) current obtained from SWV scans of zwitterionic polymer-coated gold electrodes functionalized with 5 μM of thiolated capture DNA and incubated with 1 μM of reporter DNA for 1 hour at 37° C. (n=3). (b) current obtained from SWV scans of zwitterionic polymer-coated gold electrodes coated with 5+1 μM of thiolated +aminated capture DNA and incubated with 1 μM of reporter DNA for 1 hour at 37° C. (n=3).

FIG. 14 shows limit of detection of the DNA barcode in exemplary embodiments: (a, b) in buffer using MCH-backfilled gold electrodes, (c, d) in unprocessed and undiluted human plasma using MCH-backfilled gold electrodes, (e, f) in buffer using zwitterionic polymer-coated gold electrodes, and (g, h) in unprocessed and undiluted human plasma using zwitterionic polymer-coated gold electrodes-all data points were measured in triplicate (n=3).

FIG. 15 shows detection of SARS-CoV-2 pseudovirus using DMAPS90-MAA-SH10-coated aptamer-functionalized electrodes relative to conventional PEG-backfilled aptamer-functionalized electrodes in exemplary embodiments: (a) schematic of SARS-CoV-2 pseudovirus detection using aptamer immobilized zwitterionic polymer-coated and PEG-backfilled aptamer immobilized gold electrodes; (b) R_{CT} change (%) obtained for zwitterionic polymer-coated and PEG-backfilled gold electrodes after incubation with unfiltered 50% human saliva with or without the addition of 10⁴ cp mL⁻¹ SARS-CoV-2 pseudovirus (n=3); (c) Specificity of TMSA52-immobilized zwitterionic polymer-coated electrodes for SARS-CoV-2 pseudovirus tested against 5 μL of a non-target control pseudovirus (PV) or a non-target virus (10⁴ cp or PFU mL⁻¹ in unfiltered 50% saliva) for 5 minutes at room temperature—R_{CT} change was obtained from the EIS curves before and after target incubation; the numbers below the bars show the fold change in R_{CT} change between the target (T) and blank (background (B)) of the respective comparison; non-significant (p>0.05)=ns, ** p≤0.01 obtained from two-tailed Student's t-test.

FIG. 16 shows EIS curves obtained from pre-and post-target incubation of unfiltered 50% saliva (blank) and SARS-CoV-2 pseudovirus spiked in unfiltered 50% saliva (target) in exemplary embodiments: (a) blank of DMAPS90-MAA-SH10 zwitterionic polymer-coated electrode; (b) target of zwitterionic polymer-coated gold electrode; (c) blank of PEG-backfilled gold electrode; and (d) target of PEG-backfilled gold electrode.

FIG. 17 shows storage stability of zwitterionic polymer electrodes at 4° C. (left) or room temperature (right) in exemplary embodiments: (a) the peak values of anodic current obtained from CV measurements performed in 2 mM potassium hexacyanoferrate (II) and 2 mM potassium ferricyanide solution (both prepared in 1× PBS); (b) the current peak from MB reduction obtained using SWV measurements after incubating 1 μM reporter DNA on capture DNA-immobilized zwitterionic polymer electrodes that were stored for 1-14 days-SWV measurements were performed in 25 mM NaCl: 25 mM phosphate buffer solution (25:25 buffer); non-significant (p>0.05)=ns, *p≤0.05 is obtained from a two-tailed Student's t-test between readings from Day 1 and Day 14.

FIG. 18 shows the protocol used for Antifouling RT-MagMAp. (a) Assay components involved in Antifouling RT-MagMAp. Schematic illustration of real time event progression occurring at the thiolated trimeric capture aptamer modified antifouling electrodes (b) in presence of target resulting in a sandwich formation between the trimeric aptamer on electrode and monomeric aptamer labelled with magnetic beads and (c) in absence of target where the sandwich structure is absent. ΔZ/Z signals obtained for (d) 250 pM VEGF₁₆₅ and (e) 0 pM VEGF₁₆₅ recorded for 30 minutes and reported from 2 minutes at intervals of 4 minutes.

FIG. 19 shows a schematic representation of RT-MAp assay for (a) blank (0 pM VEGF₁₆₅) and (b) target (25 pM and 2500 pM VEGF₁₆₅). (c) ΔZ/Z signals obtained for detection of 0 to 2500 pM VEGF165 spiked in 20 % plasma using RT-MAp assay. Schematic representation of RT-MagMAp assay for (d) blank (0 pM VEGF₁₆₅) and (e) target (25 pM and 2500 pM VEGF₁₆₅). (f) ΔZ/Z signals obtained for detection of 0 to 2500 pM VEGF₁₆₅ spiked in 20% plasma using RT-MagMAp assay. Schematic representation of Antifouling RT-MagMAp assay for (g) blank (0 pM VEGF₁₆₅) and (h) target (25 pM and 2500 pM VEGF₁₆₅). (i) ΔZ/Z signals obtained for detection of 0 to 2500 pM VEGF₁₆₅ spiked in 20% plasma using Antifouling RT-MagMAp assay.

FIG. 20 shows a schematic representation of RT-MAp assay for (a) blank (0 pM VEGF₁₆₅) and (b) target (25 pM and 2500 pM VEGF₁₆₅). (c) ΔZ/Z signals obtained for detection of 0 to 2500 pM VEGF₁₆₅ spiked in 20% plasma using RT-MAp assay. Schematic representation of RT-MagMAp assay for (d) blank (0 pM VEGF₁₆₅) and (e) target (25 pM and 2500 pM VEGF₁₆₅). (f) ΔZ/Z signals obtained for detection of 0 to 2500 pM VEGF₁₆₅ spiked in 20% plasma using RT-MagMAp assay. Schematic representation of Antifouling RT-MagMAp assay for (g) blank (0 pM VEGF₁₆₅) and (h) target (25 pM and 2500 pM VEGF₁₆₅). (i) ΔZ/Z signals obtained for detection of 0 to 2500 pM VEGF₁₆₅ spiked in 20% plasma using Antifouling RT-MagMAp assay.

FIG. 21 shows an RT-MAp assay for VEGF₁₆₅ detection in buffer. Each data point represents the average of measurements on three different electrodes (n=3) and the error bars represent standard deviation of the three replicates.

FIG. 22 shows optimization of aptamer concentration for RT-MAp assay for VEGF₁₆₅ detection. The error bars correspond to results obtained on replicates conducted on three different electrodes (n=3).

FIG. 23 shows optimization of on-bead concentration of trimeric aptamer for a previously optimized capture trimeric aptamer concentration of 250 nM using RT-MagMAp. Each data point represents the average of measurements on three different electrodes (n=3) and the error bars represent standard deviation of the three replicates.

FIG. 24 shows optimization of on-bead monomeric aptamer concentration for capture monomeric aptamer concentration of 1 μM using RT-MagMAp. Each data point represents the average of measurements on three different electrodes (n=3) and the error bars represent standard deviation of the three replicates.

FIG. 25 shows aptamer concentration and combination optimization using RT-MagMAp. The error bars correspond to results obtained on replicates conducted on three different electrodes (n=3).

FIG. 26 shows magnetic bead concentration optimization at optimization bead: aptamer concentration ratio for RT-MagMAp. The error bars correspond to results obtained on replicates conducted on three different electrodes (n=3).

FIG. 27 shows Poor resolution using RT-MagMAp in 20% plasma. Each data point represents the average of measurements on three different electrodes (n=3) and the error bars represent standard deviation of the three replicates.

FIG. 28 shows binding buffer proportion optimization for RT-MagMAp. The error bars correspond to results obtained on replicates conducted on three different electrodes (n=3).

FIG. 29 shows optimization of Purified RT-MagMAp. Each data point represents the average of measurements on three different electrodes (n=3) and the error bars represent standard deviation of the three replicates.

FIG. 30 shows requirement of BSA for Antifouling RT-MagMAp assay: (a) Z for BSA, (b) ΔZ/Z for BSA, (c) Z for biotin with BSA, (d) ΔZ/Z for biotin with BSA, (e) Z for no blocker, and (f) ΔZ/Z for no blocker. Each set (With polymer and without polymer) is performed once on a different single electrode (n=1).

FIG. 31 shows incorporating mutant trimeric aptamer as control for Antifouling RT-MagMAp. (a) Predicted secondary structure of the monomeric mutant VEGF165 aptamer which is transformed into a homotrimer. (b) Schematic showing dual measurement using VEGF165 specific aptamer and control mutant aptamer. (c) ΔZ/Z signals recorded for 30 minutes for different VEGF165 concentrations (0 pM to 2500 pM) spiked in 20% plasma sample using a control mutant and a VEGF165 specific trimeric aptamer. Limit of detection for Antifouling RT-MagMAp using four different calibration methods using log-linear linear-fit calibration curves obtained (d) using the maximum ΔZ/Z signal (ΔZ/Zₘₐₓ) obtained for the specific aptamer, (e) using Specific ΔZ/Z to Mutant ΔZ/Z signal ratio, (f) using Z-slope calculated between 30 minutes and 2 minutes and (g) Specific: Mutant slope calculated between 30 minutes and 2 minutes. Each sample concentration is tested in triplicates (n=3) measured on three different electrodes. The error bars represent standard deviation of the three replicates.

FIG. 32 shows optimization of thiolated trimeric aptamer concentration for Antifouling RT-MagMAp. Each data point represents the average of measurements on three different electrodes (n=3) and the error bars represent standard deviation of the three replicates.

FIG. 33 shows (a) Specific: Mutant ΔZ/Z log-linear fits at each time point with corresponding goodness of linear fit (R²) with time, showing 30 minutes to be the best fit. Each data point represents the average of measurements on three different electrodes (n=3) and the error bars represent standard deviation of the three replicates.

FIG. 34 shows test of detection specificity of VEGF165 trimeric aptamer against other common biomarkers prevalent and detectable in plasma. The solid black line represent threshold with respect to mutant aptamer calculated as ΔZ/Z signals for aptamer for 2.5 nM VEGF165 with mutant aptamer+3× standard deviation. The dotted black line represent threshold with respect to 0 nM VEGF165 with specific aptamer calculated as ΔZ/Z signals for aptamer for 2.5 nM VEGF165 with mutant aptamer+3× standard deviation. Each sample concentration is tested in triplicates (n=3) measured on three different electrodes. The error bars represent standard deviation of the three replicates. Insets show schematics illustrating the respective binding and non binding events.

FIG. 35 shows ELISA calibration to quantify plasma samples. The error bars correspond to results obtained on replicates conducted on three different wells (n=3).

FIG. 36 shows multiple plasma sample testing. (a) Specific ΔZ/Z signals recorded for 30 minutes starting at 2 minutes at intervals of 4 minutes. (b) Maximum ΔZ/Z signals (ΔZ/Zₘₐₓ) obtained for each sample. (c) Mutant ΔZ/Z signals recorded for 30 minutes starting at 2 minutes at intervals of 4 minutes. (d) Specific: Mutant ΔZ/Z obtained at 30 minutes for each sample. (e) Z-slope obtained for the samples. (f) Specific: Mutant slope obtained for the samples. Each sample is tested on once on a different electrode (n=1).

FIG. 37 shows assay quantification efficiency obtained using different calibration methods. (a) Illustration of assay operation, (b) Correlation analysis of true total VEGF₁₆₅ concentration present in tested samples to the concentration evaluated using ΔZ/Zₘₐₓ based calibration (Z), Specific: Mutant ΔZ/Z based calibration (μZ), Z-slope based calibration (m-Z) and Specific: Mutant Z-Slope based calibration (m-μZ). The plots show log transformed true vs evaluated VEGF₁₆₅ concentration along with the goodness of linear fit (R²), equation of fitted linear line showing its slope, Pearson's correlation coefficient (PCC) and p-value. (e) Log-transformed Bland-Altman plots^{[7]} comparing true total VEGF₁₆₅ concentration present in tested samples to the concentration evaluated using ΔZ/Zₘₐₓ based calibration (Z), Specific: Mutant ΔZ/Z based calibration (μZ), Z-slope based calibration (m-Z) and Specific: Mutant Z-Slope based calibration (m-μZ). (d) Summary of Bland-Altman plots^{[7]} showing the values of mean difference, limit of agreement (LOA) range, outliers and standard deviation (SD) of mean difference. 12 plasma samples with different spiked concentrations are tested with a total of 124 samples with known total concentrations. Each sample is tested once on a different electrode.

FIG. 38 shows electrode stability test: (a) ΔZ/Z signals obtained using VEGF aptamer on aptamer functionalized electrodes stored for 0, 7, 14, 21 and 28 days for sample spiked with 250 pM VEGF. (b) ΔZ/Z signals obtained using mutant aptamer on aptamer functionalized electrodes stored for 0, 7, 14, 21 and 28 days for the same sample as (a). (c) Specific: Mutant ΔZ/Z signal obtained for the electrodes tested as described in (a-b). The error bars correspond to results obtained on replicates conducted on three different electrodes (n=3).

### DETAILED DESCRIPTION

Described herein, in aspects, is a thiolated zwitterionic polymer-based coating that can facilitate adhesion to a gold electrode and efficient probe grafting using a thin polymer layer that not only retains but increases the maximum current measured via cyclic voltammetry. For example, relative to previous anti-fouling coating approaches, the polymer coating disclosed herein can (1) facilitate sufficient conductivity to enable sensing without coupled conductive/conjugated polymers, (2) anchor to the electrode surface without requiring additional adhesion mediators, (3) avoid the need to use other backfilling agents and sample pre-processing that can increase sample-to-sample variability and complexity in biosensor fabrication, and (4) provide multiple types of binding sites for diverse probe attachment, including the attachment of multiple types of probes to a single coating/electrode to maximize the probe density on the biosensor surface.

In exemplary embodiments, the coating facilitated the detection of 21 nM redox-labeled DNA in unprocessed and undiluted plasma; detection of 10⁴ cp mL⁻¹ of SARS-CoV-2 pseudovirus in unfiltered 50% saliva was also achieved with improved target-to-blank ratios and reproducibility relative to PEG-based biosensing system for detecting COVID-19.

### i. Definitions

Unless otherwise indicated, the definitions and embodiments described in this and other sections are intended to be applicable to all embodiments and aspects of the present invention herein described for which they are suitable as would be understood by a person skilled in the art. It is also to be understood that the terminology used herein is for the purpose of describing particular aspects only and is not intended to be limiting.

In understanding the scope of the present invention, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used herein, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

The term “suitable” as used herein means that the selection of the particular compound or conditions would depend on the type and purpose of the specific synthetic manipulation to be performed and the identity of the molecule(s) to be transformed as per the knowledge of one skilled in the art, including all relevant reaction conditions such as solvent, reaction time, reaction temperature, reaction pressure, reactant ratio, and requirement for inert environment reactions.

The term “sample” or “test sample” as used herein may refer to any material in which the presence or amount of a target analyte is unknown and can be determined in an assay. The sample may be from any source, for example, any biological (e.g. human or animal samples, including clinical samples), environmental (e.g. water, soil or air) or natural (e.g. plants) source, or from any manufactured or synthetic source (e.g. food or drinks). The sample may be comprised or is suspected of comprising one or more analytes. The sample may be a “biological sample” comprising cellular and non-cellular material, including, but not limited to, tissue samples, urine, blood, serum, other bodily fluids and/or secretions.

The term “target”, “analyte” or “target analyte” as used herein may refer to any agent, including, but not limited to, a small inorganic molecule, small organic molecule, metal ion, biomolecule, toxin, biopolymer (such as a nucleic acid, carbohydrate, lipid, peptide, protein), cell, tissue, microorganism and virus, for which one would like to sense or detect. The analyte may be either isolated from a natural source or is synthetic. The analyte may be a single compound or a class of compounds, such as a class of compounds that share structural or functional features. The term analyte also includes combinations (e.g. mixtures) of compounds or agents such as, but not limited, to combinatorial libraries and samples from an organism or a natural environment.

The term “nucleic acid” as used herein refers to a biopolymer comprising monomers of nucleotides, such as deoxyribonucleic acid (DNA), ribonucleic acid (RNA) and other polynucleotides of modified nucleotides and/or nucleotide derivatives and may be either double stranded (ds) or single stranded (ss). Nucleic acids may be modified to contain modified nucleotides comprising one or more modified bases (e.g. unusual bases such as inosine, and functional modifications to the bases such as amino), modified backbones (e.g. peptide nucleic acid, PNA) and/or other chemically, enzymatically, or metabolically modified forms.

The term “aptamer” as used herein refers to a short, chemically synthesized nucleic acid molecule or oligonucleotide sequence which can be generated by in vitro selection to fold into specific three-dimensional (3D) structures that bind to a specific analyte with dissociation constants, for example, in the pico-to nano-molar range. Aptamers may be single-stranded DNA, and may include RNA, modified nucleotides and/or nucleotide derivatives. Aptamers may also be naturally occurring RNA aptamers termed “riboswitches”.

The term “hybridizes”, “hybridized” or “hybridization” as used herein refers to the sequence specific non-covalent binding interaction with a complementary, or partially complementary, nucleic acid sequence. When, for example, the 5′-end region of an aptamer hybridizes to the 3′-end region, it can form a duplex DNA element.

The term “functionalizing” or “functionalized on” as used herein refers to various common approaches for functionalizing a material, which can be classified as mechanical, physical, chemical and biological. Any suitable form of coupling may be utilized (e.g. coating, binding, etc.). The functionalized material, for example, an aptamer or a blocking species, may also be immobilized.

The term “room temperature” as used herein refers to a temperature in the range of about 20° C. and about 25° C.

It will be understood that any component defined herein as being included may be explicitly excluded by way of proviso or negative limitation, such as any specific compounds or method steps, whether implicitly or explicitly defined herein.

Although methods and materials similar or equivalent to those described herein can be used in the practice or testing of this disclosure, suitable methods and materials are described below.

### ii. Polymers

Disclosed herein, in aspects, is a zwitterionic copolymer bearing sulfobetaine, carboxylic, thiol, and, optionally, aldehyde groups as a thin (about 16 nm) anti-fouling coating for electrochemical biosensing platforms. The resulting polymer-coated electrodes reduced protein adsorption by about 67% compared to the bare-gold surface when incubated with radiolabeled human serum albumin (HSA) protein-spiked human plasma, while cyclic voltammetry yielded about a 5% increase in anodic current signal after incubation in about 1% HSA for about 1 hour compared to about a 83% decrease in anodic current observed with bare gold electrodes. The polymer-coated electrode facilitated the detection of redox-labeled DNA in buffer, as well as in unprocessed and undiluted plasma with detection limits of about 23 nM and about 21 nM, respectively; detection of about 10⁴ cp mL⁻¹ lentivirus pseudotyped with the Omicron spike protein of SARS-CoV-2 in unfiltered 50% saliva was also achieved within about 5 minutes with improved target-to-blank ratios and reproducibility relative to the well-established PEG-based biosensing platform for detecting COVID-19.

A zwitterionic monomer (in a typical embodiment, DMAPS) is copolymerized with an acid-containing comonomer (in a typical embodiment methacrylic acid, MAA) and optionally an acetal-containing comonomer that can be hydrolyzed to expose aldehyde groups (Ald), with the carboxylic acid residues subsequently used to graft a thiol functional group to the polymer. The resulting DMAPS-MAA-SH (acid-containing comonomer only) has two functional groups (thiols and residual carboxylic acids) while the DMAPS-Ald-MAA-SH terpolymer has three functional groups (thiols, aldehydes and residual carboxylic acids). Thiol groups can interact with a gold electrode to anchor the polymer on the electrode surface, while both the thiol and aldehyde groups (if present) can facilitate the direct attachment of thiolated and (if aldehydes are present) aminated biorecognition elements.

Relative to other reported coating strategies, the use of this polymer to coat electrode surfaces does not typically require any blocker, spacer, or backfiller. Furthermore, unlike other reported coatings that report reduced current as a result of the added anti-fouling coating in an electrochemical biosensing context, the anodic current from CV after coating the polymer on the gold surface is higher than the current obtained from the bare gold surface; without wishing to be bound by theory, the combination of the high charge density and the thin dimensions (<20 nm thickness) of the coating enable high conductivity without requiring the inclusion of other conductive elements. Specifically, the polymer coating that can (1) facilitate sufficient conductivity to enable sensing without coupled conductive/conjugated polymers, (2) anchor to the electrode surface without additional mediators, (3) avoid the use of other backfilling agents that can increase sample-to-sample variability, and (4) provide multiple types of binding sites for diverse probe attachment, including the attachment of multiple types of probes to a single coating/electrode. Thus, the zwitterionic electrochemical biosensor coating can allow for sensitive detection of disease biomarkers/analytes while eliminating or reducing the need for sample pre-processing and/or the use of additional backfilling/blocking agents.

Accordingly, provided herein is a polymer coating for an electrochemical biosensor comprising one or more polymerizable zwitterionic monomers containing at least one cationic charge and at least one anionic charge at neutral pH and one or more first polymerizable comonomers that contains or can be modified to contain thiol groups.

Advantageously, when the coating is made in this way, there is no need for additional blockers, spacers, or backfillers in order to reduce electrode fouling.

In some embodiments, the thiol groups enable covalent, physical, or affinity binding of a sensing probe.

In some embodiments, the first polymerizable comonomer contains or can be modified to contain carboxyl groups.

In some embodiments, the polymer coating further comprises one or more second polymerizable comonomers that enables covalent, physical, or affinity binding of a sensing probe.

In some embodiments, the second polymerizable comonomer contains or can be modified to contain aldehyde groups.

In some embodiments, the thiol groups are conjugated to the carboxyl group of the first comonomer via the conjugation of a nucleophilic thiol entity selected from cysteamine, cysteine, or derivatives thereof.

In some embodiments, thiol groups are introduced via nucleophilic conjugation of a disulfide-containing small molecule. In some embodiments, thiol groups are introduced by grafting of a nucleophilic small molecule containing a disulfide group that is subsequently reduced to a thiol. In some embodiments, the nucleophilic small molecule is a dihydrazide with a disulfide in the center.

In some embodiments, the zwitterionic monomer comprises a sulfobetaine, carboxybetaine, a phosphorylcholine, or a combination thereof. In typical aspects, the zwitterionic monomer comprises [2-(methacryloyloxy)ethyl]dimethyl-(3-sulfopropyl)ammonium hydroxide (DMAPS). Other examples include 2-methacryloyloxyethyl phosphorylcholine (MPC) and carboxybetaine methacrylate (CBMA). Combinations of different monomers are also contemplated. It will be understood that the spacing between the cationic and anionic charge can vary, as can the polymerizable backbone.

In some embodiments, the first comonomer is selected from methacrylic acid (MAA), acrylic acid, vinylacetic acid, fumaric acid or maleic acid.

In some embodiments, the second comonomer is N-(2,2-dimethoxyethyl) methacrylamide (DMEMA). It will be understood, however, that the comonomer can be any comonomer that could be used to react with a functional group on a sensing probe. The skilled person would appreciate that this list would depend on what functional group is on the sensing probe and could be easily determined based on the teachings herein.

In some embodiments, the zwitterionic comonomer, such as DMAPS, comprises about 75 mol % of the total monomer residues in the polymer, the acetal-containing comonomer, such as DMEMA, comprises about 15 mol % of the total monomer residues in the polymer, and the carboxylated comonomer, such as MAA, comprises about 10 mol % of the total monomer residues in the polymer.

In some embodiments, the zwitterionic comonomer, such as DMAPS, comprises about 90 mol % of the total monomer residues in the polymer and the carboxylated comonomer, such as MAA, comprises about 10 mol % of the total monomer residues in the polymer.

In some embodiments, the coating is attached to a noble metal (e.g. gold) or noble metal (e.g. gold)-coated electrode via gold-thiol interactions. Other noble metals such as, platinum, silver, ruthenium, rhodium, palladium, osmium, iridium, and rhenium, could be used instead of gold, as will be understood. In typical aspects, however, the noble metal comprises gold. When it comes to attaching the coating to gold as described herein, it is noted that while other coatings typically have reduced current as a result of the added anti-fouling coating in an electrochemical biosensing context, in the present case, however, the anodic current from CV after coating the polymer on the gold surface is typically higher than the current obtained from the bare gold surface. In this way and in typical aspects, the coating described herein, when applied to an electrochemical biosensor, is capable of reducing fouling without compromising the current signal.

In some embodiments, the coating is less than about 20 nm in thickness. For example, in aspects, the coating is less than about 20 nm in thickness, less than about 19 nm in thickness, less than about 18 nm in thickness, less than about 17 nm in thickness or less than about 16 nm in thickness. In some embodiments, the coating is about 16 nm in thickness. For example, in aspects, the coating is about 13 nm in thickness, the coating is about 14 nm in thickness, the coating is about 15 nm in thickness, the coating is about 16 nm in thickness, the coating is about 17 nm in thickness or the coating is about 18 nm in thickness.

In some embodiments, the coating is functionalized with a sensing probe functionalized with thiol and/or amine groups. The sensing probe functionalized with thiol and/or amine groups is useful for binding to the coating.

In some embodiments, the sensing probe detects a target.

In some embodiments, the sensing probe comprises a biomolecule, synthetic affinity agent or combinations thereof. In some embodiments, the biomolecule comprise a nucleic acid. In some embodiments, the biomolecule comprise a protein. In some embodiments, the nucleic acid comprises DNA, RNA, and/or an aptamer. In some embodiments, the biomolecule is an aptamer. In some embodiments, the protein comprises an antibody or an enzyme. The sensing probe is typically multimeric, such as dimeric or trimeric.

In some embodiments, the target comprises a nucleic acid (e.g., RNA or DNA), a virus, a protein, or a combination thereof. In some embodiments, the target is DNA. In some embodiments, the biomolecule comprises a nucleic acid and the target is a complementary DNA strand, optionally labeled with an electrochemically-active group. In other embodiments, the target comprises a protein. In some embodiments, the protein comprises an antibody or an enzyme.

In some embodiments, the target is a virus. Any suitable virus may be targeted, such as, but not limited to, enteric or non-enteric viruses, enveloped or non-enveloped viruses, family selected from the group consisting of Astroviridae, Caliciviridae, Picornaviridae, Togaviridae, Flaviviridae, Caronaviridae, Paramyxviridae, Orthomyxoviridae, Bunyaviridae, Arenaviridac, Rhabdoviridae, Filoviridae, Reoviridae, Bornaviridae, Retroviridae, Poxviridae, Herpesviridae, Adenoviridae, Papovaviridae, Parvoviridae, Hepadnaviridae, (eg., a virus selected from the group consisting of a Coxsackie A-24 virus Adeno virus 11, Adeno virus 21, Coxsackie B virus, Borna Disease Virus, Respiratory syncytial virus, Parainfluenza virus, California encephalitis virus, human papilloma virus, varicella zoster virus, Colorado tick fever virus, Herpes Simplex Virus, vaccinia virus, parainfluenza virus 1, parainfluenza virus 2, parainfluenza virus 3, dengue virus, Ebola virus, Parvovirus B19 Coxsackie A-16 virus, HSV-1, hepatitis A virus, hepatitis B virus, hepatitis C virus, hepatitis D virus, hepatitis E virus, human immunodeficiency virus, Coxsackie B1-B5, Influenza viruses A, B or C, LaCross virus, Lassavirus, rubeola virus Coxsackie A or B virus, Echovirus, lymphocytic choriomeningitis virus, HSV-2, mumps virus, Respiratory Synytial Virus, Epstein-Barr Virus, Poliovirus Enterovirus, rabies virus, rubivirus, variola virus, WEE virus, Yellow fever virus and varicella zoster virus), Norwalk virus, Norovirus, Rotavirus, Astrovirus, Reovirus, coronaviruses such as SARS-CoV-1, SARS-CoV-2, and MERS-COV; influenza viruses such as H1N1, H5N1. In some embodiments, the target is a pseudovirus. In some embodiments, the biomolecule comprises an aptamer and the target is a virus or pseudovirus.

Also provided herein is an electrochemical biosensor comprising the polymer coating disclosed herein and a device comprising said electrochemical biosensor.

Also provided herein is an assay comprising an electrochemical biosensor as described herein in combination with magnetic beads. The magnetic beads typically contain a probe that is capable of detecting the target in question, thereby sandwiching the target between the magnetic beads and the polymer coating of the biosensor. This increases the sensitivity of the biosensor for detecting the target.

### EXAMPLES

The following non-limiting examples are illustrative of the present invention:

### Example 1

### Experimental

Materials. [2-(methacryloyloxy)ethyl]dimethyl-(3-sulfopropyl)ammonium hydroxide (DMAPS, 95%), methacrylic acid (MAA, 99%), ammonium persulfate (98%), thioglycolic acid (TGA, 98%), cystamine dihydrochloride (96%), 1,4-dithiothreitol (DTT, ≥97%), deuterium oxide (D₂O), sodium phosphate dibasic (≥99%), ethylenediaminetetraacetic acid (EDTA, 99.4-100.6%), 5,5′-dithiobis(2-nitrobenzoic acid) (DTNB, ≥98%), hydroxylamine hydrochloride (99%), 4-(2-hydroxyethyl)-1-piperazineethanesulfonic acid (HEPES, ≥99.5%), L-glutathione reduced (≥98%), phosphate buffer solution (1.0 M, pH 7.4), sodium chloride (NaCl, ≥99.0%), magnesium chloride (MgCl₂, ≥99.0%), tris(2-carboxyethyl)phosphine hydrochloride (TCEP), 6-mercapto-1-hexanol (MCH, ≥99%), potassium chloride (≥99.0%), potassium hexacyanoferrate (II) trihydrate (≥99.5%), hydrochloric acid (HCl, 37%), (poly(ethylene glycol) methyl ether thiol (6000 kDa), Tween-20, human serum albumin (HSA, ≥99%) were all purchased from Sigma-Aldrich (Oakville, Canada) and were used as received. Calcium chloride dihydrate (CaCl₂·2H₂O, ≥99.5%) was purchased from BioShop Canada. 1-ethyl-3-(3′-(dimethylaminopropyl)carbodiimide hydrochloride (EDAC HCl, ≥98%) was purchased from EMD Millipore. Adipic acid dihydrazide (ADH, 98%) was purchased from AlfaAcsar. Phosphate buffer saline (PBS, 10× pH 7.4) was purchased from ThermoFisher. Potassium ferricyanide (A.C.S. reagent) was purchased from Anachemia. Sulfuric acid (H₂SO₄, 98%) and 2-propanol (99.5%) were purchased from Caledon Laboratories. HPLC-purified methylene blue (MB)-labeled reporter DNA, MB-labeled polyT non-complementary reporter DNA (NC-1), and capture DNA were obtained from Integrated DNA Technologies (IDT) and used as received. Aminated non-complementary DNA sequences were obtained from IDT and then tagged with MB (NC-2 and NC-3) using MB NHS ester obtained from Glen Research (Virginia, USA), as previously reported [37]. N-(2,2-dimethoxyethyl) methacrylamide (DMEMA) was synthesized in-house, as previously reported [38]. Iodine-125 (¹²⁵I)-labeled NaI in 0.1 N sodium hydroxide (NaOH) (radiochemical purity 98%) was obtained from the McMaster Nuclear Reactor without any carrier. Human plasma and pooled human saliva were obtained from Canadian Plasma Resources (Saskatchewan, Canada) and Innovative Research Inc. (Novi, MI, USA), respectively. Spike (BA.4/5, Omicron Variant) (SARS-CoV-2) pseudotyped lentivirus was obtained from BPS Bioscience (catalog number: 78652). Control pseudovirus, influenza A (with a titer of 3.6×10⁷ PFU mL⁻¹) and adenovirus (with a titer of 1×10⁷ PFU mL⁻¹) were provided by Dr. Matthew Miller's lab at McMaster University and acquired using previously described methods [39,40]. Milli-Q grade distilled deionized water (DIW) was used for polymer synthesis, whereas autoclaved Milli-Q grade DIW was used for all biosensing experiments and buffer preparations.

Synthesis of DMAPS75-Ald15-MAA-SH10 Polymer. DMAPS (3 g, 10.7 mmol), DMEMA (372 mg, 2.1 mmol), MAA (124 mg, 1.4 mmol), ammonium persulfate (40 mg, 0.2 mmol), and TGA (10 μL) were dissolved in DIW (30 mL). The resulting solution was magnetically stirred at 350 rpm under an N2 atmosphere overnight at 75° C. to complete polymerization, after which 1 N HCl (100 mL) was added to the final product and stirred for 24 hours at room temperature to convert the acetal groups in the polymer to aldehyde groups (Ald). The aldehyde-functionalized polymer was then dialyzed in DIW using a 3.5 kDa molecular weight cutoff dialysis membrane (Spectra-Por 3 RC) (6×6 h cycles) against DIW. To thiolate the polymer, cystamine dihydrochloride (1.2 g, 5.3 mmol) was added in the dialyzed polymer, the pH was adjusted to 4.75, and EDAC HCl (1.02 g, 6.6 mmol) was added. The reaction was performed for 6 hours under 350 rpm magnetic stirring with continuous pH adjustment to maintain pH in the 4.5-5 range. The resulting graft copolymer was dialyzed (6×6 hour cycles) against DIW (pH 4.0), followed by mixing with DTT (0.31 g, 2 mmol) to cleave the disulfide bond in the grafted cystamine and expose free thiol (SH) groups. The reaction proceeded for 6 hours under 400 rpm stirring, maintaining the pH at ˜8. The pH of the resulting thiolated polymer was lowered to pH 3.5, and the polymer was dialyzed (6×6 h cycles) against 0.1 M NaCl (pH 3.5) for purification. The final product was freeze-dried and stored at 4° C.

Synthesis of DMAPS80-Ald15-MAA-SH5 Polymer. DMAPS (3 g, 10.7 mmol), DMEMA (350 mg, 2 mmol), MAA (58 mg, 0.7 mmol), APS (40 mg, 0.2 mmol), and TGA (10 μL) were dissolved in DIW (30 mL). Following, the protocol described above for DMAPS75-Ald15-MAA-SH10 was used for polymer synthesis. The thiolation procedure also followed the same general protocol but instead using cystamine dihydrochloride (0.61 g, 2.7 mmol), EDAC HCl (0.52 g, 3.3 mmol), and dithiothreitol (0.16 g, 1.0 mmol) amounts that account for the lower targeted thiol content in this polymer.

Synthesis of DMAPS80-MAA-SH20 Polymer. DMAPS (3 g, 10.7 mmol), MAA (230 mg, 2.7 mmol), APS (40 mg, 0.2 mmol), and TGA (10 μL) were dissolved in DIW (30 mL) and polymerized under 350 rpm magnetic stirring under an N₂ atmosphere overnight at 75° C. The resulting polymer was dialyzed against DIW using a 3.5 kDa cutoff dialysis membrane (6×6 hour cycles) and lyophilized. Following, to thiolate the synthesized polymer, cystamine dihydrochloride (2.4 g, 10.7 mmol) was added into the dialyzed polymer. The pH was adjusted to 4.75, after which EDAC HCl (2.04 g, 13.1 mmol) was added and the reaction was allowed to proceed for 6 hours under 350 rpm magnetic stirring (maintaining the pH between 4.5-5 throughout the reaction by adding 0.1 M HCl or NaOH as required). Following, the resulting polymer was dialyzed (6×6 h cycles) and dithiothreitol (0.62 g, 4 mmol) was added to cleave the disulfide bonds and expose free thiol groups (400 rpm stirring, pH ˜8). After 6 hours of stirring, the pH was changed to pH 3.5, and the sample was dialyzed against 0.1 M NaCl adjusted to pH 3.5 using 1 M HCl (6×6 h cycles). The final product was freeze-dried and stored at 4° C.

Synthesis of DMAPS90-MAA-SH10 Polymer. DMAPS (3 g, 10.7 mmol), MAA (103 mg, 1.2 mmol), APS (40 mg, 0.2 mmol), and TGA (10 μL) were dissolved in DIW (30 mL) and polymerized as described for the synthesis of DMAPS80-MAA-SH20. A similar protocol was used to thiolate the synthesized polymer but using cystamine dihydrochloride (1.20 g, 5.3 mmol), EDAC HCl (1.02 g, 6.6 mmol) and dithiothreitol (0.31 g, 2 mmol) amounts reflective of the lower target thiol content.

Characterization of Polymers. The chemical structure of the polymers was characterized by ¹H NMR using a Bruker AVANCE 600 MHz spectrometer. For the analysis, 10 mg of the polymer was dissolved in 1 mL of D₂O solvent. Polymer molecular weight was measured using an Agilent 1260 Infinity II GPC system with an Agilent 1260 infinity refractive index detector and a Superose 6 Increase 10/300 GL (GE Healthcare) column maintained at 30° C. The polymer was analyzed prior to the acid hydrolysis and thiolation steps to avoid undesirable polymer-column interactions. 10 mg of the freeze-dried polymer was dissolved in 1 mL of 1× PBS solution containing 0.05% sodium azide and kept in a shaker for 24 hours at 37° C. and 100 rpm. Afterward, the solution was filtered through a 0.2 um sterile filter, loaded into the GPC column with a flow rate of 0.5 mL min-1, and calibrated with narrow PEG standards (molecular weights 3-60 kDa). Hydroxylamine titration for assessing aldehyde content was performed using Burivar-I2 automatic buret (ManTech Associates) [41]. 0.25 M hydroxylamine hydrochloride solution was prepared in DIW, and 100 mg of the polymer was dissolved in 50 mL of the prepared hydroxylamine solution. The volume of 0.1 M NaOH needed to return the solution pH 4.0 was recorded, with the mol % of free aldehydes in the polymer calculated relative to the titrant volume required to titrate a DMAPS90-MAA10 polymer control in which the aldehyde comonomer was fully replaced with DMAPS to account for any potential interferences. To assess the free thiol content of the polymers following DTT treatment, Ellman's analysis was performed [42]. 150 mL of Ellman's assay buffer (EAB) was prepared using ethylenediaminetetraacetic acid (55.8 mg, 0.2 mmol) and sodium phosphate dibasic (2.13 g, 15 mmol) to adjust the pH of the buffer to ˜8-8.5. DTNB solution was also prepared by dissolving DTNB (4 mg, 0.01 mmol) in 1 mL of prepared EAB. Thiol contents were measured by mixing 1 mL of EAB, 50 μL of DTNB, and 250 μL of a 0.5 wt % polymer solution in DIW. Each sample was measured in triplicate using a VICTOR 3 multi-label microplate reader reading at an absorbance at 405 nm. The thiol content was calculated based on a calibration curve prepared using L-GSH as the standard (0-4.4 mM concentration).

Fabrication of Polymer-Coated Gold Electrodes. Gold electrodes were fabricated on polystyrene sheets (Graphix Shrink Film, Graphix) pre-cleaned with 2-propanol and DIW followed by air drying. A vinyl mask (FDC 4304, FDC Graphic Films) patterned for the required electrode shape using Adobe Illustrator and a Robo Pro CE5000-40-CRP cutter (Graphtec America) was then attached to the cleaned polystyrene sheet and used as a mask to sputter a 100 nm gold film using DC sputtering (MagSput, Torr International). The mask was removed, after which the overall electrode (FIG. 6) except the working area (diameter 2 mm and geometric surface area of 3.14 mm²), was covered with a vinyl mask and cleaned with 2-propanol and DIW followed by electrochemical cleaning through cyclic voltammetry (CV) in 0.1 M H₂SO₄ (0-1.5 V, 100 mVs⁻¹ and 40 cycles) using a potentiostat (PalmSens4, PalmSens BV, Netherlands), with Ag/AgCl as the reference electrode and platinum wire as the counter electrode. The electroactive area of the gold electrodes was calculated by dividing the electrochemical charge obtained from the area under the reduction curve with the surface charge density of gold oxide monolayer formation (482 μC cm⁻²). The cleaned chips (with mask applied) were then dip-coated over a 24 hour period in a fresh polymer solution prepared by dissolving 30 mg of polymer in 1 mL of autoclaved DIW, mixing with 0.39 M TCEP to ensure the presence of free thiols, and dialyzing out excess TCEP using 3.5 kDa membrane for 2 hours against 10 mM NaCl under 80 RPM stirring, after which the dialyzed product was diluted to the required concentration of 4 mg mL⁻¹ using DIW and 0.75 mL of 10× PBS to achieve a final volume of 7.5 mL in 1× PBS. After 24 hours of dipping, the electrodes were removed from the polymer solution and gently wicked dried with a Kimwipe, after which the mask covering the other parts of the electrode was removed. After removing the mask, the electrodes were dried at room temperature for 1.5 hours and then washed with autoclaved DIW twice by gentle dipping, wicked dry with a Kimwipe, air dried, and stored at room temperature until further use.

Characterization of the Polymer-Coated Gold Electrode. Contact angles were determined in triplicate for bare and polymer-coated gold surfaces prepared with different polymer concentrations (0.1, 4, and 8 mg mL⁻¹) using a KRUSS DSA30S DropShape Analyzer (Hamburg, Germany). Droplets of 1× PBS (10 μL) were used as the test solution. The thickness of polymer film on the gold surface was measured using a UV-vis ellipsometer (J.A. Woollam M-2000, wavelength range 246-1688 nm) operating at five different incident angles from 55° to 75° with an interval of 5°. A 2×2 cm² piece of 4 mg mL⁻¹ polymer-coated gold on silicon was used for the measurements. The accompanying COMPLETE EASE software was used to estimate the coating thickness by fitting the model with the least mean square error (MSE) to the obtained Psi and Delta values at different angles. The presence of polymer and accessibility of the aldehyde groups present coated on the gold surface was confirmed using the hydrazide-functionalized fluorescent dye fluorescein-thiosemicarbazide (FTSC). The dye was dissolved in DMSO, diluted with DIW to make a 0.05 mg mL⁻¹ solution, incubated on a 4 mg mL⁻¹ polymer-coated gold surface for 2 hours at room temperature, and washed vigorously in water. The residual fluorescence was observed using an optical microscope (Nikon, Minato, Tokyo, Japan) with a Nikon blue excitation fluorescence filter and compared to controls of a gold surface without a polymer coating and with DMAPS90-MAA-SH10 polymer coating (neither of which have aldehyde groups). Electrochemical characterization of polymer-coated gold electrodes was performed using cyclic voltammetry (CV) scans at varying scan rates (potential range: 0-0.4 V, scan rate: 10 to 200 mV s⁻¹, cycles: 2), using 4 mg mL⁻¹ polymer-coated gold electrodes in 2 mM potassium hexacyanoferrate (II) and 2 mM potassium ferricyanide solution (prepared in 1× PBS). Similarly, CV was performed on bare and 4 mg mL⁻¹ polymer-coated gold electrodes in 2 mM potassium hexacyanoferrate (II) and 2 mM potassium ferricyanide solution at a scan rate of 50 mV s⁻¹ to compare the anodic current of polymer-coated gold electrodes with bare gold electrodes.

Protein Adsorption for Anti-Fouling. Protein adsorption to the coated and uncoated electrodes was assessed using radiolabeled HSA-¹²⁵I protein spiked in plasma. 1% HSA-¹²⁵I was prepared using the iodine monochloride method [43,44] as follows: 1 mL of a 10 mg mL⁻¹ HSA solution in 1× PBS was mixed with 0.2 mL of 2 M glycine buffer (pH 8.8) at a volumetric ratio of 5:1 (Vial A). Vial B was prepared by mixing 0.178 mL of 0.0033 M iodine monochloride (ICl) reagent (prepared in-house [45]) with 0.891 mL of 2 M glycine buffer at a volumetric ratio of 1:5. 10 μL of ¹²⁵I-labeled NaI (1000 μCi) in 0.1 M NaOH was subsequently added to vial B, left to mix for 3 minutes, and then mixed with the contents of vial A for an additional 5 minutes. Afterwards, the radiolabeled protein was passed through a syringe column packed with AG1-X4 resin (Bio-Rad, Hercules, CA, USA) to remove any unbound free-¹²⁵I followed by rinsing the column with 5 mL of 1× PBS. The collected radiolabeled protein was tested for free-¹²⁵I using trichloroacetic acid precipitation [46], with the free ¹²⁵I content confirmed to be below 3%. The obtained 1.71 mg mL⁻¹ of radiolabeled HSA-¹²⁵I protein was diluted in human plasma to yield a 1% HSA-¹²⁵I solution (0.4 mg mL⁻¹ radiolabeled protein in 40 mg mL⁻¹ HSA in human plasma). To test protein adsorption to electrode surfaces, 3 μL of the human plasma solution was added to the electrodes and incubated for 1 hour at room temperature on bare gold or 4 mg mL⁻¹ polymer-coated electrodes, after which the electrodes were washed three times in 1× PBS to remove any loosely bound protein. The electrodes were then wicked dry with a Kimwipe, loaded on counting vials, and inserted into a Wizard 3 1480 Automatic Gamma Counter (Perkin Elmer, Waltham, MA, USA) to measure the radioactivity of the adsorbed protein on the electrodes. Standards of working solution and negative control (1× PBS) were also prepared to convert the obtained counts into μg of proteins. The obtained amount of protein was normalized using the electroactive area of gold electrodes used for the assay.

Electrochemical Characterization of Anti-Fouling. To assess anti-fouling from an electrochemical perspective, 1% HSA (10 mg mL⁻¹ in 1× PBS) was deposited on the bare gold electrode as well as polymer-coated electrodes prepared by dip coating the electrode in 4 mg mL⁻¹ polymer solution and incubating at room temperature for 24 hours. After washing the electrodes in 1× PBS and autoclaved DIW twice, CV in 2 mM potassium hexacyanoferrate (II) and 2 mM potassium ferricyanide solution (prepared in 1× PBS) was performed (potential range: 0-0.4 V, scan rate: 50 mV s⁻¹, cycles: 2). The anodic current (μA) from the CV curves was used to compare the anti-fouling properties.

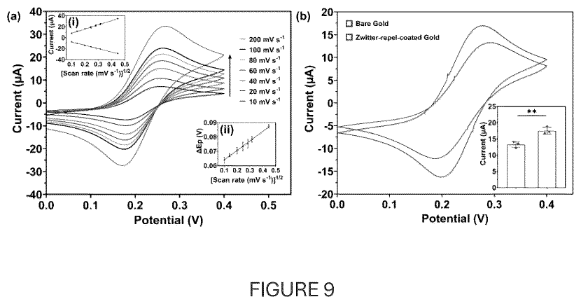

Polymer-Coated Gold Electrode Biofunctionalization. 4 mg mL⁻¹ DMAPS75-Ald15-MAA-SH10 polymer-coated gold electrodes were incubated with varying concentrations of aminated and/or thiolated capture DNA for 18 hours at room temperature in the dark using 25 mM NaCl:25 mM phosphate buffer solution: 100 mM MgCl₂ (25:25:100) as the immobilization buffer; the majority of experiments were conducted at a concentration of 5+1 μM of thiolated+aminated capture DNA. After the incubation, electrodes were washed with 25 mM NaCl: 25 mM phosphate buffer solution (25:25 buffer) and then hybridized with 1 μM MB-labeled reporter DNA (in 25:25:100 buffer with 0.001% Tween-20) for 1 hour at 37° C. to allow for hybridization between the capture DNA and the reporter DNA. Square wave voltammetry (SWV) measurements were subsequently conducted in 25:25 buffer (potential range: 0 to-0.6 V, amplitude: 25 mV, frequency: 250 Hz) to record the methylene blue reduction signal. Hybridization buffer was used as a negative control for the study. CV and electrochemical impedance spectroscopy (EIS) measurements (frequency range: 0.1-100000 Hz, applied potential (E_{dc}): 0.25 V as chosen from the half-wave potential of CV curve obtained using bare-gold electrode (FIG. 9 (b))) were recorded after polymer coating and capture DNA immobilization to check the quality of the fabricated electrodes before use. A Randles circuit comprising the electrolyte resistance (R_{electrolyte}), charge transfer resistance (R_{CT}), Warburg element (W), and double layer capacitance (C_{dl}) was used for extracting R_{CT} values by fitting the obtained EIS curves using PSTrace v5.9 software. Zwitterionic polymer-coated electrodes were also incubated with 1 μM of three non-complementary reporter DNA (NC-1, NC-2, and NC-3) to test for specificity. The oligonucleotides used in this study are summarized in Table 3.

Determination of the Limit of Detection (LOD) and Recovery of Reporter DNA spiked in Undiluted and Unprocessed Human Plasma. LOD was determined using both 4 mg mL⁻¹ DMAPS75-Ald15-MAA-SH10 polymer-coated gold and bare gold electrodes immobilized with 5+1 μM of thiolated+aminated capture DNA and 1 μM thiolated capture DNA, respectively. Gold electrodes with 1 μM thiolated capture DNA were further backfilled with 100 mM MCH for 10 minutes in the dark. Both polymer and gold electrodes were washed in 25:25 buffer, after which the prepared electrodes were hybridized with different concentrations of methylene blue-labeled reporter DNA (0-1000 nM in hybridization buffer) spiked in buffer, and in unprocessed and undiluted human plasma for 1 hour at 37° C. SWV measurements were used to record the methylene blue reduction signals at different concentrations of reporter DNA (potential range: 0 V to −0.6 V, amplitude: 25 mV, frequency: 250 Hz), with all measurements performed in triplicate; error bars represent the standard deviation. LOD was calculated after determining the limit of blank (LOB), as summarized in Equations 1 and 2:

$$LOB = Average current {signal}_{blank} + 3 * {σ}_{blank} \qquad ( Equation 1 )$$

$$LOD = LOB + 3 * {σ}_{lowest detectable concentration} \qquad ( Equation 2 )$$

where σ represents the standard deviation and the factor 3 was used to calculate LOD within a 99.7% confidence interval. True LOD was then calculated by fitting the obtained LOD value using the regression equation [47].

To measure the recovery of the spiked targets, MB-labeled reporter DNA at different concentrations (35, 75, 250, and 500 nM) was added to human plasma samples and incubated on 5+1 μM of thiolated+aminated capture DNA-immobilized zwitterionic polymer-coated electrodes (n=3) for 1 hour at 37° C. After washing the electrodes in 25:25 buffer, SWV measurements were recorded and the obtained current values were fitted to the equation y=0.5433x−0.6942 (the LOD equation for zwitterionic polymer-coated electrodes in plasma) to determine the concentration of reporter DNA added. The recovery percentage and the relative standard deviation (RSD) were calculated as per Equations 3 and 4:

$$Recovery \% = \frac{Reporter DNA recovered}{Reporter DNA added} * 100 \qquad ( Equation 3 )$$

$$RSD = \frac{Recovery \%}{Standard deviation} * 100. \qquad ( Equation 4 )$$

Detection of SARS-CoV-2 Pseudovirus Spiked in Unprocessed Human Saliva. Thiolated TMSA52 aptamer specific to the spike protein of SARS-CoV-2 virus was used to detect SARS-CoV-2 pseudovirus spiked in unfiltered 50% saliva [48]. To prepare a polymer-coated functional electrode, 1 μM thiolated TMSA52 was mixed with 100 μM TCEP in 25:25:100 buffer and deposited on a 4 mg mL⁻¹ DMAPS90-MAA-SH10 polymer-coated electrode for 18 hours at room temperature. To prepare a bare gold functional electrode, the electrode was coated with 1 μM thiolated TMSA52 and backfilled with 1 mM thiolated-PEG prepared in 1× BBT buffer (50 mM HEPES, 6 mM KCl, 150 mM NaCl, 2.5 mM CaCl₂·2H₂O, 2.5 mM MgCl₂, 0.01% Tween-20, pH 7.4) as described by Zhang et al [49]. SARS-CoV-2 pseudovirus was then spiked in unfiltered 50% saliva (diluted with 1× BBT buffer) and deposited on the electrodes for 5 minutes at room temperature (consistent with prior work [49]), after which the electrodes were washed in 1× BBT buffer; control experiments were also performed using only unfiltered 50% saliva. EIS scans (frequency range: 0.1-20000 Hz, Ede: 0.25 V) were then run before and after SARS-CoV-2 pseudovirus deposition, with negative controls performed in 2 mM potassium hexacyanoferrate (II) and 2 mM potassium ferricyanide solution with 50 mM KCl in 1× PBS. Randles circuit was fitted to the obtained EIS curves to calculate the charge transfer resistance (R_{CT}). The % R_{CT} change for EIS scan before and after depositing the SARS-CoV-2 pseudovirus sample was calculated for both types of electrodes in unfiltered 50% saliva (Equation 5), while the fold change was calculated by dividing the % R_{CT} change following SARS-CoV-2 pseudovirus addition in unfiltered 50% saliva with the % R_{CT} change observed with the respective negative control (Equation 6):

$$\% {R}_{CT} change = \frac{{R}_{CTf} - {R}_{CTi}}{{R}_{CTi}} * 100 \qquad ( Equation 5 )$$

$$Fold change = \frac{\% {R}_{CT} {change}_{T}}{\% {R}_{CT} {change}_{B}} . \qquad ( Equation 6 )$$

Herein, R_{CTf} is charge transfer resistance after sample incubation followed by washing, R_{Cti} is the charge transfer resistance before sample incubation, % R_{CT} change_{T} is the % R_{CT} change following incubation of sample with SARS-CoV-2 pseudovirus (target), and % R_{CT} changes is the % R_{CT} change obtained from incubation in the same buffer but without added SARS-CoV-2 pscudovirus (blank). The prepared TMSA52-immobilized DMAPS90-MAA-SH10 polymer-coated electrodes were also tested against a control pseudovirus (which lacks the SARS-CoV-2 spike protein but is otherwise the same compositionally) and two real viruses (influenza A and adenovirus) for specificity testing at a concentration of 10⁴ cp mL⁻¹ (pseudovirus) or PFU mL⁻¹ (virus) in unfiltered 50% saliva.

Storage Stability Studies. Three zwitterionic polymer-coated electrodes were stored in a petri dish in vacuum-sealed bags at either 4° C. or room temperature. One day before the stability test, the electrodes were removed from the bag and their anodic current was measured using CV (potential range: 0-0.4 V, scan rate: 50 mV s−1) in 2 mM potassium hexacyanoferrate (II) and 2 mM potassium ferricyanide solution (both prepared in 1× PBS). The electrodes were then immobilized with 5+1 μM of thiolated+aminated capture DNA. On the day of testing, the electrodes were washed in 25:25 buffer twice by gentle dipping, incubated with 1 μM of MB-labeled reporter DNA for 1 hour at 37° C., and tested using SWV for storage stability assessment.

Statistical Analysis. Two-tailed student's t-test was performed to assess statistical significance between groups. The groups with p-value less than 0.05 were considered significant. All the experiments were performed with a sample size of 3, and the bar plots represent mean±standard deviation. The analysis and graphs were plotted in GraphPad Prism 10.

### Results and Discussion

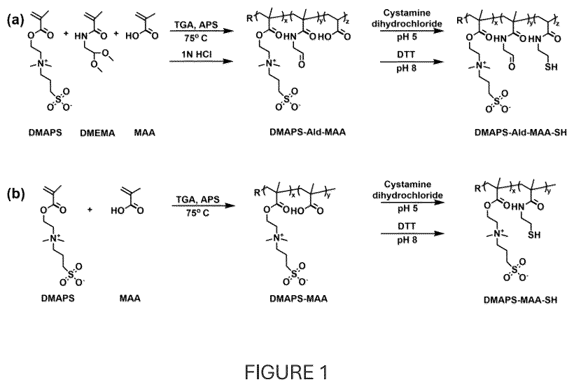

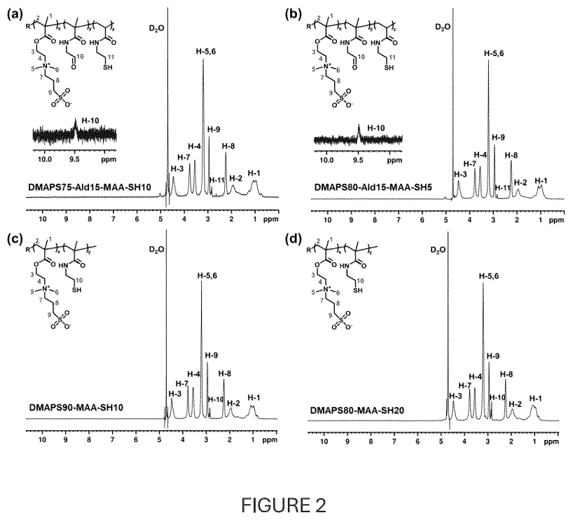

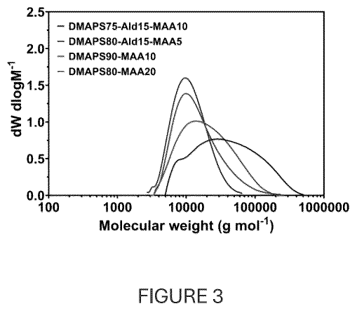

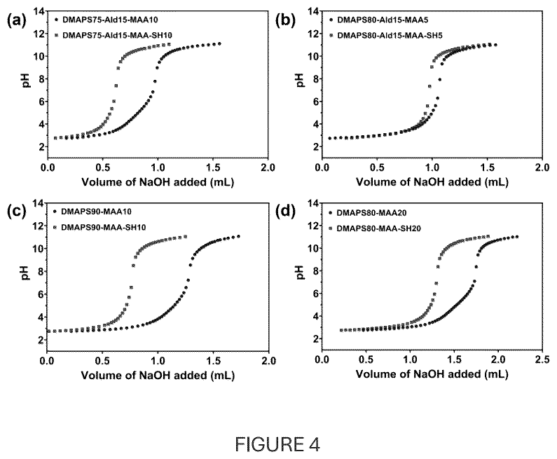

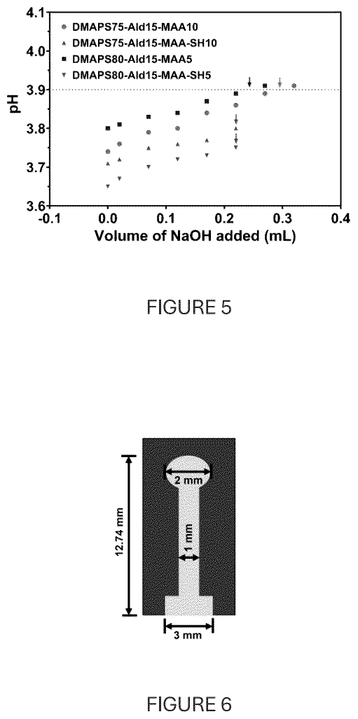

Polymer Characterization. The DMAPS-aldehyde comonomer-methacrylic acid (DMAPS-Ald-MAA) and DMAPS-methacrylic acid (DMAPS-MAA) precursor polymers were synthesized using free radical copolymerization and subsequently thiolated by grafting cystamine to the carboxylic acid groups present in the polymer using a carbodiimide-mediated reaction (FIG. 1). ¹H-NMR (FIG. 2) confirmed the presence of the expected functional groups in each polymer, particularly the aldehyde signal at ˜9.5 ppm in polymers containing an aldehyde group. Table 1 summarizes the quantitative chemical characterization of the copolymers. The synthesized polymers exhibited relatively low molecular weights in the range of 9.6-23.2 kDa with dispersities in the range of 1.4-2.7 as determined by aqueous gel permeation chromatography (FIG. 3). Base-into-acid conductometric titration indicated near-quantitative incorporation of MAA into the polymers (±5 mol % of the targeted —COOH content, FIG. 4), while hydroxylaminc hydrochloride titration indicated near-quantitative aldehyde functionalization in the DMAPS-Ald-MAA terpolymers that was only minimally changed (via the potential formation of thioacctals) following thiol functionalization (FIG. 5). Conductometric titration indicated that 50-70% of available —COOH groups were functionalized with thiol groups, with the lack of quantitative conversion likely a result of steric inhibition of some free —COOH groups following the initial grafting reaction; as such, the polymers retain a low (3.9-6.7 mol %) fraction of residual carboxylated comonomer residues (FIG. 4). Complementarily, Ellman's assay indicated that concentration of free thiols was 30-55% lower than the measured graft yield of the thiol conjugation reaction (Table 1 and Table 2), indicative of the formation of a corresponding portion of disulfides within the polymers. Regardless of potential disulfide formation, the final polymers have scalable free thiol contents to enable gold electrode immobilization as well as tunable aldehyde contents to promote aminated probe immobilization.

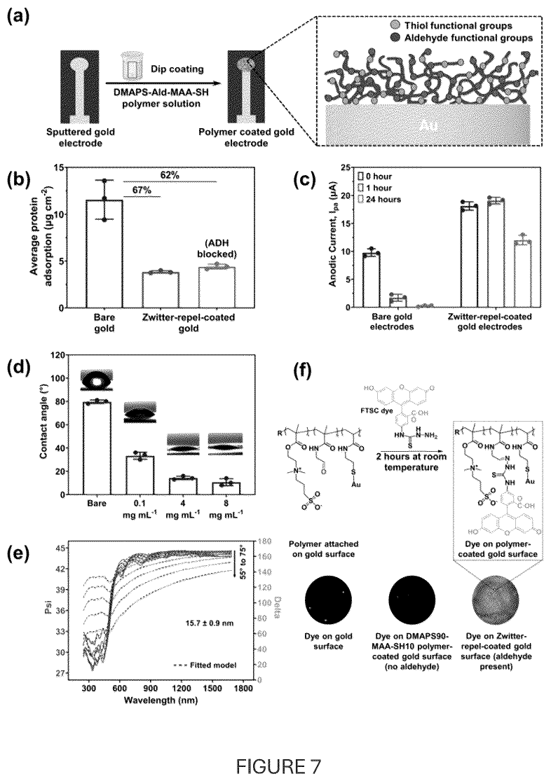

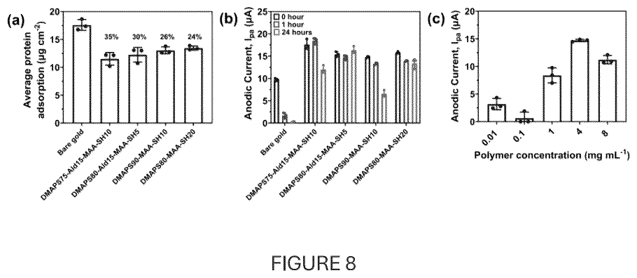

Electrode Coating with Anti-Fouling Polymers. The synthesized functional zwitterionic polymers were subsequently coated on the surface of a cleaned gold electrode (with an electroactive arca of 2.1±0.1 mm²) by dip coating the electrodes in an aqueous polymer solution (FIG. 7 (a)). Although disulfides have also been observed to bind to gold surfaces (albeit with low coverage) [50], TCEP was used to reduce any disulfides that form in the precursor polymers into free thiols immediately before coating on gold electrodes and thus optimize binding affinity of the polymer to the electrode surface. To assess the anti-biofouling properties of the coatings with varying polymers and polymer concentrations, protein adsorption was screened against 2% radiolabeled HSA-1251 protein-spiked human plasma (FIG. 8). All zwitterionic polymers reduced the adsorbed protein content between 24% to 35%, with the DMAPS75-Ald15-MAA-SH10 polymer showing the highest anti-fouling efficiency (FIG. 8 (a)). Similar trends were observed based on the anodic current obtained from the CV scans before and after 1% HSA incubation over 24 hours (FIG. 8 (b)); although the DMAPS80-Ald15-MAA-SH5 polymer showed slightly higher maintenance of the anodic current, the DMAPS75-Ald15-MAA-SH10 polymer optimized in the radiolabeling study also retained a large percentage of the anodic current signal. Anodic current was also generally maximized at higher polymer concentrations, although the 4 mg mL⁻¹ dip-coated samples retained significantly higher anodic current relative to other tested concentrations (FIG. 8 (c)). As such, in consideration of both the radioactivity adsorption study and the CV study results, the DMAPS75-Ald15-MAA-SH10 polymer (hereafter referred to as the zwitterionic polymer) was chosen for subsequent biosensing studies and dip coated on electrodes at a polymer concentration of 4 mg mL⁻¹ unless otherwise indicated.

To more explicitly compare the anti-fouling properties of the zwitterionic polymer with those of the bare gold electrodes, a series of experiments was conducted as summarized in FIG. 7. First, 1% HSA-¹²⁵I protein-spiked human plasma was incubated with both bare and polymer-coated electrodes for 1 hour. The average protein adsorption to bare gold electrodes was 11.6±2.1 μg cm⁻² whereas electrodes coated with zwitterionic polymer adsorbed 3.8±0.1 μg cm⁻² of protein (FIG. 7 (b)), representing a 67% decrease in protein adsorption due to the presence of the coating. To assess if the free aldehyde groups (present to enable subsequent probe grafting) significantly hinder the protein-repellent nature of the coating via potential Schiff base formation with proteins, ADH was used to block the free aldehyde groups. The resulting protein adsorption was 4.4±0.3 μg cm⁻² (FIG. 7 (b)), representing a similar 62% decrease in protein adsorption relative to the bare gold electrode as observed prior to aldehyde blocking; as such, the presence of aldehyde groups at the concentration present in the optimized polymer does not significantly impact the protein-repellent properties of the coating. A similar result is again obtained via electrochemical analysis following incubation of the electrodes in 1% HSA solution (FIG. 7 (c)). After 1 hour and 24 hours of incubation with HSA, the anodic currents on the bare gold electrodes were reduced by 83% and 97%, respectively, while there was no significant change in current after 1 hour and only a 37% decrease in current after 24 hours of incubation with the zwitterionic polymer-coated electrode. This result is consistent with the significant decrease in contact angle observed upon polymer coating (FIG. 7 (d)), showing a reduction from ˜80° with the bare gold electrode to ˜15° with the 4 mg mL⁻¹ polymer-coated electrode. Ellipsometry measurements confirmed the presence of the polymer coating on the electrodes (FIG. 7 (e)), indicating a thin film thickness of ˜16 nm. Collectively, these results confirm the presence of the zwitterionic polymer coating on the electrode and its potential to impart significant anti-fouling properties to the electrode surface.

The presence and accessibility of free aldehyde groups (for probe grafting) following the coating procedure was assessed by incubating the electrode in FTSC dye, which can form a hydrazone bond with free aldehyde groups and thus impart fluorescence to the bound surface. No fluorescence was observed in the absence of polymer or when a polymer without aldehyde groups (DMAPS90-MAA-SH10) was coated on the gold surface; in contrast, strong green fluorescence was observed for DMAPS75-Ald15-MAA-SH10 (the zwitterionic polymer)-coated gold electrodes, confirming the presence of free and accessible aldehyde groups on the gold surface following coating (FIG. 7 (f)).

To demonstrate the potential functionality of the zwitterionic polymer coating for electrochemical biosensing, CV scans were performed on the zwitterionic polymer-coated electrodes. CV scans performed at different scan rates (FIG. 9 (a)) showed the expected redox peaks, with the magnitude of the peaks increasing linearly with the square root of scan rate indicative of a diffusion-controlled charge transport process on the polymer-coated gold electrodes (FIG. 9 (a) inset i) as well as the plot between peak potential difference and the square root of scan rate (FIG. 39 (a) inset ii) shows the quasi-reversible nature of the redox reaction occurring at the electrode surface. Interestingly, in contrast to other reported zwitterionic coatings, a higher anodic peak current was observed with the polymer-coated gold electrodes (17.6 ±1.1 μA) compared to the bare gold electrodes (13.3 ±0.98 μA) (FIG. 9 (b)), suggesting that the zwitterionic polymer coating not only avoids insulating the surface but actually promotes charge transfer. This increase in anodic current for zwitterionic polymer-coated gold electrodes can be attributed to the high charge concentration of the zwitterionic polymer (including the residual —COOH groups not grafted with thiols) coupled with the thin dimensions (˜16 nm thickness, FIG. 7 (e)) of the coating, resulting in a thin and highly hydrated polymer layer that contrasts with other thicker and/or denser coatings that have been reported. Furthermore, the demonstrated capacity of zwitterionic materials to promote the formation of ion channels [51] coupled with the stronger self-interaction observed with sulfobetaine polymers (weakening ion pairing with other competing ions in solution [52]) collectively result in enhanced mobility of the highly charged potassium ferro/ferricyanide ions (4K⁺[Fe(CN)₆]⁴⁻and 3K⁺[Fe(CN)₆]³⁻) toward the active surface area of the electrode, resulting in higher apparent ferro/ferricyanide signal.

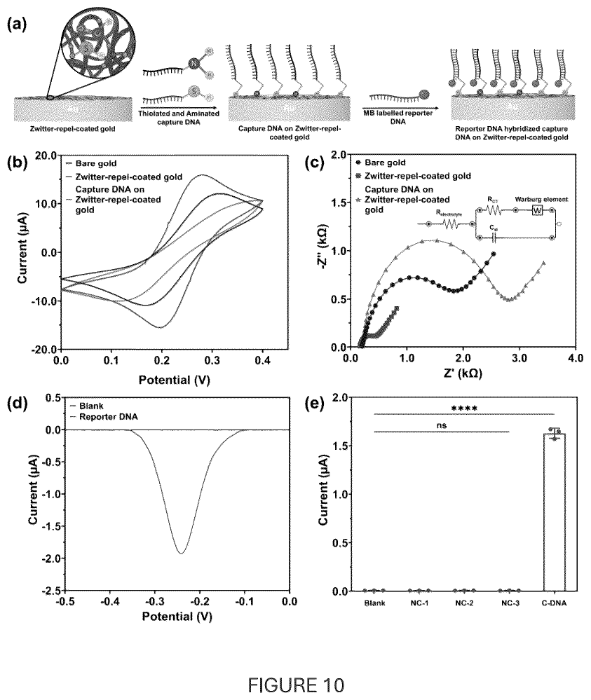

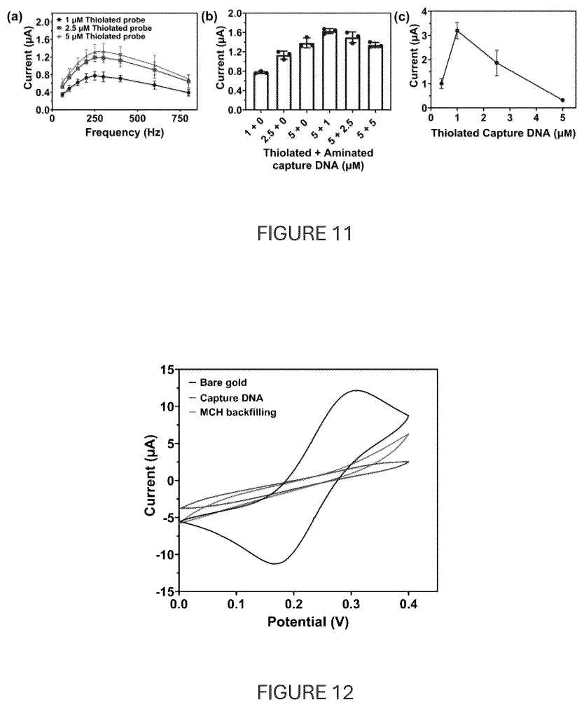

Detection of Complementary Electrochemically Active DNA Probes. To assess the potential of the polymer-coated electrodes for biosensing, each bare gold electrode was electrochemically characterized with CV and EIS after coating with both polymer and a combination of thiolated and aminated capture DNA, taking advantage of the dual thiol/aldehyde functionalization of the coating to maximize probe density on the electrode surface (FIG. 10 (a)). Optimization of different SWV frequencies ranging from 10 to 800 Hz indicated that 250 Hz yielded the highest current signal obtained from MB reduction at all capture DNA concentrations tested (FIG. 11 (a)); as such, this frequency was used for all subsequent measurements. Screening of different concentrations of thiolated and aminated capture DNA subsequently incubated with 1 μM of MB-labeled reporter DNA for 1 hour indicated that a combination of 5+1 μM of thiolated+aminated capture DNA in which the thiolated capture DNA attaches to the gold surface and/or the free thiol groups on the polymer chain and the aminated capture DNA attaches to the aldehyde groups of the polymer gave the highest retained current signal based on the reduction of MB during SWV scans (FIG. 11 (b)); consequently, that probe combination was selected for subsequent studies. In contrast, the maximum current achieved with the bare gold electrode following incubation in various concentrations of thiolated capture DNA was achieved at just 1 μM probe concentration (FIG. 11 (c)). The use of both thiolated and aminated DNA probes allowed both the gold electrode and the 3D polymer coating to be modified with probe, allowing the zwitterionic polymer coating to enable a significant increase in the amount of the capture barcode grafted to the electrode interface and thus a potentially larger dynamic range for sensing.

CV curves showed that the optimized zwitterionic polymer coating increases the anodic current of the gold surface from 12.4±0.3 μA to 16.3±0.6 μA (FIG. 10 (b)), consistent with the previous result showing improved surface charge transfer following zwitterionic polymer coating (FIG. 9 (b)). Following deposition of the optimized concentration of 5+1 μM thiolated+aminated capture DNA, the current decreases to 6.7±0.7 μA (FIG. 10 (b)), consistent with the insulating properties of DNA probes and thus confirming capture DNA deposition. Of note, the retained anodic current following probe grafting in this system is significantly higher than that achieved on a bare gold electrode prepared with the optimized 1 μM thiolated DNA concentration and backfilled with MCH (FIG. 12), a result attributable to the increase in the number of functional groups available within the thin polymer coating that can enable significantly higher attachment of capture DNA while only minimally altering the charge transfer ability. In parallel, the R_{CT} of the bare gold electrode extracted through the Nyquist plot obtained from EIS scans was reduced from 2.6±0.1 kΩ to 0.34±0.05 kΩ after polymer deposition, with the subsequent increase in charge transfer resistance following probe deposition to 8.5±1.2 kΩ correlating directly to the results obtained from CV (FIG. 10 (c)).

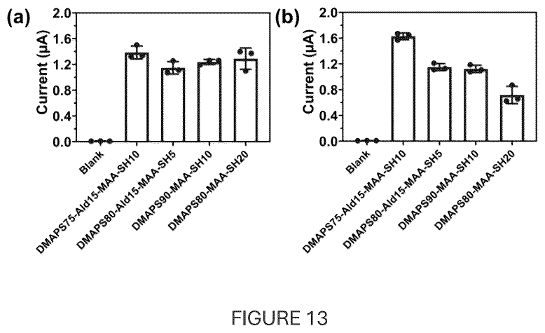

Methylene blue is a benchmark reporter commonly used in electrochemical biosensors, with several reports published on detecting MB-labeled DNA barcodes by DNA hybridization with capture DNA attached to the electrode surface due to the complementarity between both DNA sequences [53-57]. The zwitterionic polymer-coated electrodes functionalized with the optimized dual thiolated (5 μM)/aminated (1 μM) DNA probes were subsequently tested for selectively detecting a DNA barcode complementary to the probe DNA that contains a redox reporter (methylene blue) for generating an electrochemical signal. In the presence of complementary barcode (reporter) DNA, SWV shows a large reduction peak associated with the reduction of methylene blue; in contrast, the peak is completely absent when no DNA is added (FIG. 10 (d)). Similarly, no significant electrochemical signal was observed upon the incubation of three non-complementary DNA reporters (NC-1, NC-2, NC-3) individually with capture-DNA immobilized zwitterionic polymer-coated electrodes (FIG. 10 (e)). These results confirm the successful modification of the electrode with probe DNA and the resultant specificity of biosensor to detect a complementary DNA sequence. Of note, if only 5 μM thiolated capture probe (no aminated probe) was used, electrodes coated with a variety of different polymer compositions showed similar SWV current signals when incubated with 1 μM reporter DNA (FIG. 13 (a)); however, when the polymer surfaces were coated with a combination of thiolated and aminated capture DNA (5+1 μM), the DMAPS75-Ald15-MAA-SH10 polymer facilitated a significantly higher current signal in comparison to other polymers (FIG. 13 (b)). This result confirms the benefit of the dual thiol/aldehyde functionality on the optimized zwitterionic polymer for promoting higher DNA probe attachment despite the marginally better electrochemical anti-fouling performance of the lower thiol content DMAPS80-Ald15-MAA-SH5 polymer (FIG. 13 (b)).

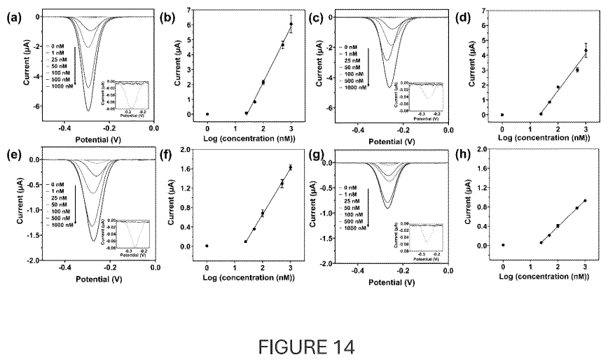

To further assess the efficiency of the prepared electrodes to detect reporter DNA in complex media at low concentrations, the LOD of the fabricated polymer electrodes was determined by plotting the peak current signal obtained from the SWV measurements against varying reporter DNA concentrations (0-1000 nM) spiked in either buffer or 100% unprocessed and undiluted plasma followed by performing a linear regression; bare gold electrodes coated with probe DNA and backfilled with MCH were used as a control (FIG. 14). LOD values for MCH-backfilled capture DNA immobilized gold electrodes were 27 nM in spiked buffer and 23 nM in spiked plasma samples (FIG. 14 (a-d)), while LOD values of 23 nM in spiked buffer and 21 nM in spiked plasma samples were observed for the zwitterionic polymer-coated electrodes (FIG. 14 (e-h)). The similar LOD values obtained for both systems showed that the polymer coating does not inhibit the performance of conductive biosensors while still providing high specificity of responses in complex media. Of note, the ˜16 nm thick layer zwitterionic polymer results in more hybridization of the MB-DNA further from the electrode surface, resulting in less responsive SWV signals; however, despite this, the LOD performance is similar and the standard deviation in the replicate signals is lower (FIG. 14) compared to conventional MCH backfilling. The use of the zwitterionic polymer also saves a step in the biosensor fabrication process by avoiding the need to backfill while replacing the use of small molecule backfilling agents (e.g. MCH) that are typically corrosive and/or eye/skin irritants with a non-toxic and cytocompatible polymer, both of which would be anticipated to improve the scalability and consistency of biosensor fabrication.

Table 4 shows the recovery of reporter DNA spiked in undiluted and unprocessed human plasma samples. The recovery distribution for the four samples ranged from 95.1 to 100.1% with an RSD ranging from 2.6 to 10.4%, performance on par with existing assays for different target analytes using a variety of techniques (e.g. ELISA [58,59], turbidimetry [60,61], or electrochemiluminescence [19,62]). This indicates the potential for using zwitterionic polymer-coated electrodes for detection in complex real samples.

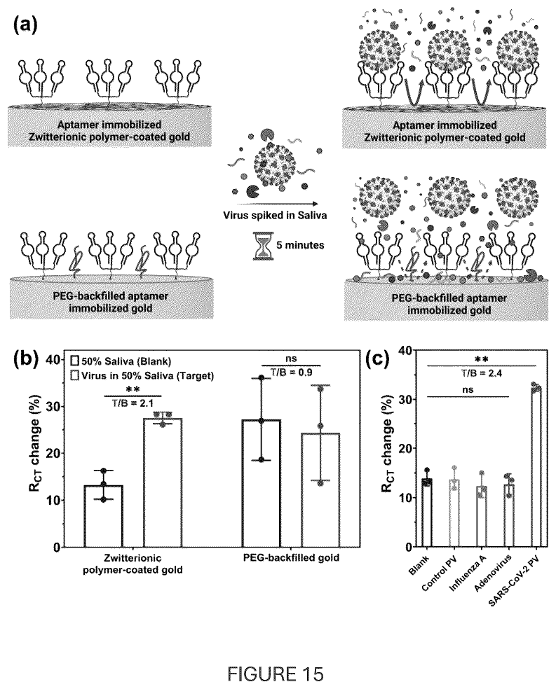

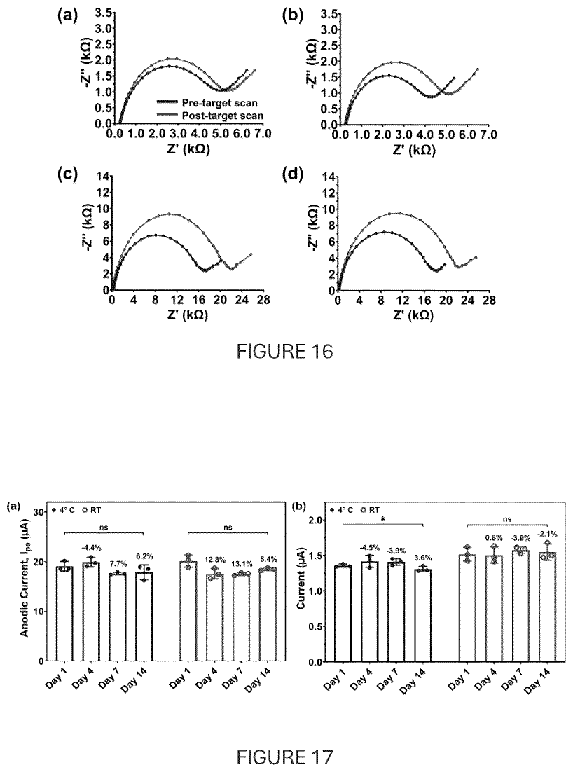

Detection of SARS-CoV-2 Pseudovirus Spiked in Unprocessed Human Saliva. To assess the capacity of zwitterionic polymer-coated electrodes for detecting unlabeled target analytes in unprocessed complex media, detection of a pseudotyped lentivirus that expresses the spike protein of the omicron variant of SARS-CoV-2 (simply termed SARS-CoV-2 pseudovirus in this study) in unfiltered 50% saliva using a previously reported trimeric aptamer probe prepared in-house (TMSA52) [48] was carried out using EIS (FIG. 15 (a); see FIG. 16 for the raw EIS curves). For this study, DMAPS90-MAA-SH10 was used as the coating polymer as only the thiolated aptamer was available for conjugation, making the presence of the aldehyde groups redundant to the coating design. Based on the established higher capacity for probe binding to the polymer-coated electrodes (FIG. 11 (a-b)), 1 μM thiolated trimeric TMSA52 was applied to both polymer-coated electrodes and bare gold electrodes. The bare gold electrodes were backfilled with thiolated PEG, while the zwitterionic polymer-coated electrodes were used directly without any subsequent backfilling. After 5 minutes of incubation with SARS-CoV-2 pseudovirus at room temperature (a time chosen based on previous studies [49]), PEG-backfilled electrodes showed a target-to-blank ratio of 0.9 in unfiltered 50% saliva in response to 10⁴ cp mL⁻¹ SARS-CoV-2 pseudovirus; in contrast, the zwitterionic polymer-coated electrodes achieved a target-to-blank ratio of 2.1 (FIG. 15 (b)). In addition, the zwitterionic polymer-coated electrodes showed significantly lower blank signals, significantly lower sample-to-sample variability (as reflected by the much smaller magnitude of the error bars), and effective detection of the target in unfiltered 50% saliva, the latter of which was not possible with the PEG-backfilled electrodes. Of note, the R_{CT} changes obtained upon the incubation of a non-specific pseudovirus as well as the real viruses influenza A and adenovirus (all also tested at concentrations of 10⁴ cp or PFU mL⁻¹ in unfiltered 50% saliva) were all found to be similar to the blank (FIG. 15 (c)), confirming the specificity of the prepared electrodes to the SARS-CoV-2 pseudovirus. Overall, the zwitterionic polymer family of coatings shows clear benefits in terms of facilitating highly effective anti-fouling properties (FIG. 7 (b)) without compromising the surface conductivity required for effective electrochemical biosensing (FIG. 7 (c)) or detection time (FIG. 15 (a)) while also avoiding the extensive sample pre-processing (at minimum centrifugation) often required with previously reported anti-fouling coatings (Table 5). The zwitterionic polymer coatings also do not require any backfilling agents (e.g. MCH), blocking agents (e.g. monoethanolamine (MEA), bovine serum albumin (BSA)) or linkers (e.g. MUA, PBA) to achieve relevant biosensing properties in saliva or blood plasma, making the coatings both simpler and safer to fabricate than conventional electrochemical biosensors. The zwitterionic polymer coatings are also significantly more efficient than PEG-based coatings when used to detect SARS-CoV-2 pseudovirus in unfiltered 50% saliva, showing a clear advantage relative to existing leading biosensor designs. Similar advantages could be achieved with a range of other types of biorecognition targets, with the dual thiol/amine reactivity of the zwitterionic polymer coating making the coating more directly amenable to grafting of a larger diversity of potential recognition elements without requiring additional modification chemistry.

Storage Stability. Throughout a 14-day storage period at either 4° C. or room temperature, no significant change in the measured anodic CV current was observed (FIG. 17 (a)). Similarly, less than 5% change in the SWV current signal was observed following the further incubation of 1 μM reporter DNA for 1 hour at 37° C. (FIG. 17 (b)), with the room temperature result again showing no significant change in current reported after 14 days. As such, the prepared electrodes are highly stable (even upon room temperature storage) for at least 14 days post-fabrication.

### Conclusions

Disclosed herein is the use of a multi-functional (thiol, carboxyl, and optionally, aldehyde group) zwitterionic copolymer as an anti-fouling electrode coating for enabling electrochemical target analyte detection in complex biological media without requiring any backfilling, blocking, or sample pre-processing steps. Thin, hydrophilic, and ionically conductive films can be formed on gold electrodes following a simple dip coating process that can significantly improve the anti-fouling properties of the electrode without negatively impacting (and in many cases improving) charge transfer at the electrode surface. The capture DNA-immobilized zwitterionic polymer electrodes showed similar behavior as the gold standard MCH-based system when tested with different concentrations of MB-labeled reported DNA spiked in buffer, as well as in unprocessed and undiluted plasma while also being able to detect 10⁴ cp mL⁻¹ SARS-CoV-2 pseudovirus in unfiltered 50% saliva within 5 minutes of incubation, performance not achievable with conventional PEG-based backfilled electrodes reported for related assays. The zwitterionic polymer electrodes can be similarly applied for enabling effective electrochemical biosensing of other diseases/biomarkers present in complex media without requiring any type of sample pre-processing while at the same time simplifying the electrode fabrication process.

### Tables

TABLE 1
Chemical characterization of zwitterionic polymer coatings.

| | | | | | | | | | | Free thiol |
|---|---|---|---|---|---|---|---|---|---|---|
| | | | | | | | Aldehyde titration | | | content via |
| | | | | —COOH titration | | Experimental | | Experimental aldehyde | | Ellman's |
| | Molecular | | | Experimental | | thiol | | (mol monomer | | analysis |
| | Weight | | Theoretical | COOH (mol %) | | (mol % | Theoretical | residues %) | | (mol % |
| | Mₙ | | COOH | Before | After | monomer | aldehyde | Before | After | monomer |
| Polymer | (kDa) | Ð | (mol %) | thiolation | thiolation | residues) | (mol %) | thiolation | thiolation | residues) |
| DMAPS80-MAA-SH20 | 14.3 | 1.9 | 20 | 17.2 | 6.7 | 10.5 | — | — | — | 6.4 |
| DMAPS80-Ald15- | 9.6 | 1.4 | 5 | 9.0 | 4.0 | 5 | 15 | 12.4 | 11.1 | 3.4 |
| MAA-SH5 | | | | | | | | | | |
| DMAPS90-MAA-SH10 | 11.5 | 1.7 | 10 | 15.4 | 6.2 | 9.2 | — | — | — | 4.2 |
| DMAPS75-Ald15- | 23.2 | 2.7 | 10 | 13.5 | 3.9 | 9.6 | 15 | 14.4 | 10.7 | 6.4 |
| MAA-SH10 | | | | | | | | | | |

TABLE 2
Free thiol content of polymers obtained from Ellman's analysis.

| | | Equation (from L- | | Free thiol content |
|---|---|---|---|---|
| | Absorbance at | GSH standard | Concentration | (mol % monomer |
| Polymer | 405 nm (y) | calibration curve) | (mM) | residues) |
| DMAPS75-Ald15-MAA- | 0.394 | y = 1.5772x − 0.002 | 0.251 | 6.4 |
| SH10 | | (R² = 1) | | |
| DMAPS80-Ald15-MAA- | 0.196 | y = 1.4412x + 0.0122 | 0.128 | 3.4 |
| SH5 | | (R² = 0.9998) | | |
| DMAPS90-MAA-SH10 | 0.158 | y = 1.6321x + 0.0031 | 0.156 | 4.2 |
| | | (R² = 0.9997) | | |
| DMAPS80-MAA-SH20 | 0.380 | y = 1.4412x + 0.0122 | 0.255 | 6.4 |
| | | (R² = 0.9998) | | |

TABLE 3
Summary of the oligonucleotides used.

| Name | Labels | Sequence (5′-3′) |
|---|---|---|
| Aminated capture | 3′-amine | TAGCTAGGAAGAGTCACACA-amine |
| DNA | | |
| Thiolatedcapture DNA | 3′-thiol | TAGCTAGGAAGAGTCACACA-thiol |
| Methylene blue- | 5′-MB | MB-TTTTTTGTGTGACTCTTCCTAGCTA |
| labelled reporter | | |
| DNA | | |
| (Complementary) | | |
| Non-complementary | 5′-MB | MB-TTTTTTTTTTTTTTTTTTTTTTTTT |
| reporter DNA | | |
| TMSA52 | 3′-thiol | TTACGTCAAGGTGTCACTCCTAGGGTTTG |
| | | GCTCCGGGCCGGCGTCGGTCGTCTCTCGC |
| | | GAAGCATCTCTTTGGCGTGTTTTTTTTTT TTTTT- |
| | | Trebler-TTTTT-thiol |

TABLE 4
Analytical results for reporter DNA in unprocessed
and undiluted human plasma samples.

| Sample | Reporter DNA | Reporter DNA | Recovery | RSD |
|---|---|---|---|---|
| No. | added (nM) | found (nM)^{#} | (%) | (%) |
| 1 | 500.0 | 492.3 | 98.5 | 10.4 |
| 2 | 250.0 | 250.3 | 100.1 | 8.1 |
| 3 | 75.0 | 73.3 | 97.7 | 2.6 |
| 4 | 35.0 | 33.3 | 95.1 | 4.7 |

^{#}Average of three measurements

TABLE 5
Comparison with previously reported anti-fouling polymers used for electrochemical biosensing.

| Anti-fouling | Target analyte | | | | | Additional steps |
|---|---|---|---|---|---|---|
| biosensing | and detection | | Time of | Complex | Anti-fouling | (Linker/Blocker/ |
| platform | method | LOD | detection | media | properties | Backfiller) |
| Thiol- | PSA (EIS) | LOD: <1 ng | 60 minutes | Not | <1% change | MUA used to |
| terminated | | mL⁻¹ (in | | tested | in R_{CT} | attach |
| sulfobetaine | | buffer only) | | | with 67 μg | biorecognition |
| on gold [31] | | | | | mL⁻¹ HSA for | element via |
| | | | | | 1 hour | EDC/NHS |
| | | | | | | Blocking with |
| | | | | | | ethanolamine |
| PPC with | TNF-α | LOD: 10 pg | 100 minutes | Whole | Negligible | PBA used for |
| PBA on | (Chrono- | mL⁻¹ (in | | blood | change in | attachment with |
| ITO [34] | amperometry) | buffer only) | | | R_{CT} with 1 | biorecognition |
| | | | | | mg mL⁻¹ | element using |
| | | | | | HSA for 1 | EDC/NHS |
| | | | | | hour | |
| PDA- | Carcinoembryonic | LOD: 3.3 fg | 90 minutes | Human | ~25% | PDA used for |
| pSBMA on | antigen (CEA) | mL⁻¹ (in | | serum | decrease in | attachment with |
| GCE [35] | (Differential | buffer only) | | | DPV signal | biorecognition |
| | pulse | | | | with 10% | elements |
| | voltammetry | | | | human serum | |
| | (DPV)) | | | | sample over | |
| | | | | | 20 minutes | |
| Catechol and | Adenosine | LOD: 0.01 | 120 minutes | 1% human | ~45% increase | PDA and AgNCs |
| zwitterion- | triphosphate | pM (in | | serum | in R_{CT} with | used for |
| bifunctionalized | (ATP) (EIS) | buffer only) | | samples | 10% human | attachment with |
| poly(ethylene | | | | | plasma samples | b-PEG and |
| glycol) | | | | | and ~18% | biorecognition |
| (b-PEG) on | | | | | increase in | element |
| GCE [63] | | | | | R_{CT} with 1 | b-PEG used as a |
| | | | | | mg mL⁻¹ | backfiller for |
| | | | | | lysozyme | anti-fouling |
| | | | | | and BSA | properties |

### References

[1] N. Liu, Z. Xu, A. Morrin, X. Luo, Low fouling strategies for electrochemical biosensors targeting disease biomarkers, Anal. Methods 11 (2019) 702-711. https://doi.org/10.1039/c8ay02674b.
[2] M. Labib, E. H. Sargent, S. O. Kelley, Electrochemical Methods for the Analysis of Clinically Relevant Biomolecules, Chem. Rev. 116 (2016) 9001-9090. https://doi.org/10.1021/acs.chemrev.6b00220.
[3] P.-H. Lin, B.-R. Li, Antifouling strategies in advanced electrochemical sensors and biosensors, Analyst 145 (2020) 1110-1120. https://doi.org/10.1039/C9AN02017A.
[4] A. Warsinke, A. Benkert, F. W. Scheller, Electrochemical immunoassays, Fresenius. J. Anal. Chem. 366 (2000) 622-634. https://doi.org/10.1007/s002160051557.
[5] S. Campuzano, M. Pedrero, P. Yáñez-Sedeño, J. Pingarrón, Antifouling (Bio) materials for Electrochemical (Bio) sensing, Int. J. Mol. Sci. 20 (2019) 423. https://doi.org/10.3390/ijms20020423.
[6] B.L. Hanssen, S. Siraj, D. K. Y. Wong, Recent strategies to minimise fouling in electrochemical detection systems, Rev. Anal. Chem. 35 (2016) 1-28. https://doi.org/10.1515/revac-2015-0008.
[7] M.J. Russo, M. Han, P. E. Desroches, C. S. Manasa, J. Dennaoui, A. F. Quigley, R. M. I. Kapsa, S. E. Moulton, R. M. Guijt, G. W. Greene, S. M. Silva, Antifouling Strategies for Electrochemical Biosensing: Mechanisms and Performance toward Point of Care Based Diagnostic Applications, ACS Sensors 6 (2021) 1482-1507. https://doi.org/10.1021/acssensors.1c00390.
[8] C. Jiang, G. Wang, R. Hein, N. Liu, X. Luo, J.J. Davis, Antifouling Strategies for Selective In Vitro and In Vivo Sensing, Chem. Rev. 120 (2020) 3852-3889. https://doi.org/10.1021/acs.chemrev.9b00739.
[9] D. Tan, F. Li, B. Zhou, Antifouling Self-Assembled Monolayers for Designing of Electrochemical Biosensors, Int. J. Electrochem. Sci. 15 (2020) 9446-9458. https://doi.org/10.20964/2020.09.56.
[10] N. Bunyakul, A. J. Bacumner, Combining Electrochemical Sensors with Miniaturized Sample Preparation for Rapid Detection in Clinical Samples, Sensors 15 (2014) 547-564. https://doi.org/10.3390/s150100547.
[11] E. Cesewski, B. N. Johnson, Electrochemical biosensors for pathogen detection, Biosens. Bioelectron. 159 (2020) 112214. https://doi.org/10.1016/j.bios.2020.112214.
[12] R. Miranda-Castro, R. Sánchez-Salcedo, B. Suárez-Álvarez, N. De-los-Santos-Álvarez, A. J. Miranda-Ordieres, M. Jesús Lobo-Castañón, Thioaromatic DNA monolayers for target-amplification-free electrochemical sensing of environmental pathogenic bacteria, Biosens. Bioelectron. 92 (2017) 162-170. https://doi.org/10.1016/j.bios.2017.02.017.
[13] V. Dharuman, J.H. Hahn, Label free electrochemical DNA hybridization discrimination effects at the binary and ternary mixed monolayers of single stranded DNA/diluent/s in presence of cationic intercalators, Biosens. Bioelectron. 23 (2008) 1250-1258. https://doi.org/10.1016/j.bios.2007.11.015.
[14] V. Dharuman, B.-Y. Chang, S.-M. Park, J. H. Hahn, Ternary mixed monolayers for simultaneous DNA orientation control and surface passivation for label free DNA hybridization electrochemical sensing, Biosens. Bioclectron. 25 (2010) 2129-2134. https://doi.org/10.1016/j.bios.2010.02.019.
[15] V. Dharuman, K. Vijayaraj, S. Radhakrishnan, T. Dinakaran, J. S. Narayanan, M. Bhuvana, J. Wilson, Sensitive label-free electrochemical DNA hybridization detection in the presence of 11-mercaptoundecanoic acid on the thiolated single strand DNA and mercaptohexanol binary mixed monolayer surface, Electrochim. (2011) 8147-8155. https://doi.org/10.1016/j.electacta.2011.05.115.
[16] O. Y. F. Henry, J. L. A. Sanchez, C. K. O'Sullivan, Bipodal PEGylated alkanethiol for the enhanced electrochemical detection of genetic markers involved in breast cancer, Biosens. Bioelectron. 26 (2010) 1500-1506. https://doi.org/10.1016/j.bios.2010.07.095.
[17] D.-S. Shin, Z. Matharu, J. You, C. Siltanen, T. Vu, V. K. Raghunathan, G. Stybayeva, A. E. Hill, A. Revzin, Sensing Conductive Hydrogels for Rapid Detection of Cytokines in Blood, Adv. Healthc. Mater. 5 (2016) 659-664. https://doi.org/10.1002/adhm.201500571.
[18] N. Hui, X. Sun, S. Niu, X. Luo, PEGylated Polyaniline Nanofibers: Antifouling and Conducting Biomaterial for Electrochemical DNA Sensing, ACS Appl. Mater. Interfaces 9 (2017) 2914-2923. https://doi.org/10.1021/acsami.6b11682.
[19] L. Ma, S. Jayachandran, Z. Li, Z. Song, W. Wang, X. Luo, Antifouling and conducting PEDOT derivative grafted with polyglycerol for highly sensitive electrochemical protein detection in complex biological media, J. Electroanal. Chem. 840 (2019) 272-278. https://doi.org/10.1016/j.jelechem.2019.04.002.
[20] G. Wang, R. Han, X. Su, Y. Li, G. Xu, X. Luo, Zwitterionic peptide anchored to conducting polymer PEDOT for the development of antifouling and ultrasensitive electrochemical DNA sensor, Biosens. Bioelectron. 92 (2017) 396-401. https://doi.org/10.1016/j.bios.2016.10.088.
[21] M. Cui, Y. Wang, M. Jiao, S. Jayachandran, Y. Wu, X. Fan, X. Luo, Mixed Self-Assembled Aptamer and Newly Designed Zwitterionic Peptide as Antifouling Biosensing Interface for Electrochemical Detection of alpha-Fetoprotein, ACS Sensors 2 (2017) 490-494. https://doi.org/10.1021/acssensors.7b00103.
[22] Y. Wang, M. Cui, M. Jiao, X. Luo, Antifouling and ultrasensitive biosensing interface based on self-assembled peptide and aptamer on macroporous gold for electrochemical detection of immunoglobulin E in serum, Anal. Bioanal. Chem. 410 (2018) 5871-5878. https://doi.org/10.1007/s00216-018-1201-9.
[23] E. González-Fernández, M. Staderini, N. Avlonitis, A. F. Murray, A. R. Mount, M. Bradley, Effect of spacer length on the performance of peptide-based electrochemical biosensors for protease detection, Sensors Actuators B Chem. 255 (2018) 3040-3046. https://doi.org/10.1016/j.snb.2017.09.128.
[24] Q. Shao, S. Jiang, Influence of Charged Groups on the Properties of Zwitterionic Moieties: A Molecular Simulation Study, J. Phys. Chem. B 118 (2014) 7630-7637. https://doi.org/10.1021/jp5027114.
[25] Y. Zhang, Y. Liu, B. Ren, D. Zhang, S. Xic, Y. Chang, J. Yang, J. Wu, L. Xu, J. Zheng, Fundamentals and applications of zwitterionic antifouling polymers, J. Phys. D. Appl. Phys. 52 (2019) 403001. https://doi.org/10.1088/1361-6463/ab2cbc.
[26] S. Liu, J. Tang, F. Ji, W. Lin, S. Chen, Recent Advances in Zwitterionic Hydrogels: Preparation, Property, and Biomedical Application, Gels 8 (2022) 46. https://doi.org/10.3390/gels8010046.
[27] L. Zheng, H. S. Sundaram, Z. Wei, C. Li, Z. Yuan, Applications of zwitterionic polymers, React. Funct. Polym. 118 (2017) 51-61. https://doi.org/10.1016/j.reactfunctpolym.2017.07.006.
[28] K. Qu, Z. Yuan, Y. Wang, Z. Song, X. Gong, Y. Zhao, Q. Mu, Q. Zhan, W. Xu, L. Wang, Structures, properties, and applications of zwitterionic polymers, ChemPhysMater 1 (2022) 294-309. https://doi.org/10.1016/j.chphma.2022.04.003.
[29] T. Goda, M. Tabata, M. Sanjoh, M. Uchimura, Y. Iwasaki, Y. Miyahara, Thiolated 2-methacryloyloxyethyl phosphorylcholine for an antifouling biosensor platform, Chem. Commun. 49 (2013) 8683. https://doi.org/10.1039/c3cc44357d.
[30] T. Bertok, L. Klukova, A. Sediva, P. Kasák, V. Semak, M. Micusik, M. Omastova, L. Chovanová, M. Vlček, R. Imrich, A. Vikartovska, J. Tkac, Ultrasensitive Impedimetric Lectin Biosensors with Efficient Antifouling Properties Applied in Glycoprofiling of Human Scrum Samples, Anal. Chem. 85 (2013) 7324-7332. https://doi.org/10.1021/ac401281t.
[31] P. Jolly, N. Formisano, J. Tkáč, P. Kasák, C. G. Frost, P. Estrela, Label-free impedimetric aptasensor with antifouling surface chemistry: A prostate specific antigen case study, Sensors Actuators B Chem. 209 (2015) 306-312. https://doi.org/10.1016/j.snb.2014.11.083.
[32] Y.-S. Wang, S. Yau, L.-K. Chau, A. Mohamed, C.-J. Huang, Functional Biointerfaces Based on Mixed Zwitterionic Self-Assembled Monolayers for Biosensing Applications, Langmuir 35 (2019) 1652-1661. https://doi.org/10.1021/acs.langmuir.8b01779.
[33] A. L. Gui, E. Luais, J. R. Peterson, J. J. Gooding, Zwitterionic Phenyl Layers: Finally, Stable, Anti-Biofouling Coatings that Do Not Passivate Electrodes, ACS Appl. Mater. Interfaces 5 (2013) 4827-4835. https://doi.org/10.1021/am400519m.
[34] C. Jiang, M. T. Alam, S. M. Silva, S. Taufik, S. Fan, J. J. Gooding, Unique Sensing Interface That Allows the Development of an Electrochemical Immunosensor for the Detection of Tumor Necrosis Factor a in Whole Blood, ACS Sensors 1 (2016) 1432-1438. https://doi.org/10.1021/acssensors.6b00532.
[35] Z. Xu, R. Han, N. Liu, F. Gao, X. Luo, Electrochemical biosensors for the detection of carcinoembryonic antigen with low fouling and high sensitivity based on copolymerized polydopamine and zwitterionic polymer, Sensors Actuators B Chem. 319 (2020) 128253. https://doi.org/10.1016/j.snb.2020.128253.
[36] H. Wu, C.-J. Lec, H. Wang, Y. Hu, M. Young, Y. Han, F.-J. Xu, H. Cong, G. Cheng, Highly sensitive and stable zwitterionic poly(sulfobetaine-3,4-ethylenedioxythiophene) (PSBEDOT) glucose biosensor, Chem. Sci. 9 (2018) 2540-2546. https://doi.org/10.1039/C7SC05104B.
[37] A. Scott, R. Pandey, S. Saxena, E. Osman, Y. Li, L. Soleymani, A Smartphone Operated Electrochemical Reader and Actuator that Streamlines the Operation of Electrochemical Biosensors, ECS Sensors Plus 1 (2022) 014601. https://doi.org/10.1149/2754-2726/ac5fb3.
[38] D. Sivakumaran, E. Bakaic, S. B. Campbell, F. Xu, E. Mueller, T. Hoare, Fabricating degradable thermoresponsive hydrogels on multiple length scales via reactive extrusion, microfluidics, self-assembly, and electrospinning, J. Vis. Exp. 2018 (2018) 1-12. https://doi.org/10.3791/54502.
[39] K. H. D. Crawford, R. Eguia, A. S. Dingens, A. N. Loes, K. D. Malone, C. R. Wolf, H. Y. Chu, M. A. Tortorici, D. Veesler, M. Murphy, D. Pettie, N. P. King, A. B. Balazs, J. D. Bloom, Protocol and Reagents for Pseudotyping Lentiviral Particles with SARS-CoV-2 Spike Protein for Neutralization Viruses 12 (2020) 513. Assays, https://doi.org/10.3390/v12050513.
[40] W. He, G. S. Tan, C. E. Mullarkey, A. J. Lec, M. M. W. Lam, F. Krammer, C. Henry, P. C. Wilson, A. A. Ashkar, P. Palese, M. S. Miller, Epitope specificity plays a critical role in regulating antibody-dependent cell-mediated cytotoxicity against influenza A virus, Proc. Natl. Acad. Sci. 113 (2016) 11931-11936. https://doi.org/10.1073/pnas.1609316113.
[41] M. A. Campea, A. Lofts, F. Xu, M. Yeganch, M. Kostashuk, T. Hoare, Disulfide-Cross-Linked Nanogel-Based Nanoassemblies for Chemotherapeutic Drug Delivery, ACS Appl. Mater. Interfaces 15 (2023) 25324-25338. https://doi.org/10.1021/acsami.3c02575.
[42] G.L. Ellman, Tissue sulfhydryl groups, Arch. Biochem. Biophys. 82 (1959) 70-77. https://doi.org/10.1016/0003-9861 (59) 90090-6.
[43] A. S. McFARLANE, Efficient Trace-labelling of Proteins with Iodine, Nature 182 (1958) 53-53. https://doi.org/10.1038/182053a0.
[44] D. M. Doran, I. L. Spar, Oxidative iodine monochloride iodination technique, J. Immunol. Methods 39 (1980) 155-163. https://doi.org/10.1016/0022-1759 (80) 90304-X.
[45] L. Liu, T. Rambarran, B. Muirhead, F. Lasowski, H. Sheardown, A Radiolabeling Method for Preciso Quantification of Polymers, Bioconjug. Chem. 33 (2022) 634-642. https://doi.org/10.1021/acs.bioconjchem.2c00047.
[46] B. Hall, M. Heynen, L. W. Jones, J. A. Forrest, Analysis of Using I 125 Radiolabeling for Quantifying Protein on Contact Lenses, Curr. Eye Res. (2015) 1-10. https://doi.org/10.3109/02713683.2015.1031350.
[47] D. A. Armbruster, T. Pry, Limit of blank, limit of detection and limit of quantitation., Clin. Biochem. Rev. 29 Suppl 1 (2008) S49-52. http://www.ncbi.nlm.nih.gov/pubmed/18852857.
[48] J. Li, Z. Zhang, J. Gu, R. Amini, A. G. Mansfield, J. Xia, D. White, H. D. Stacey, J. C. Ang, G. Panesar, A. Capretta, C. D. M. Filipe, K. Mossman, B. J. Salena, J. B. Gubbay, C. Balion, L. Solcymani, M. S. Miller, D. Yamamura, J. D. Brennan, Y. Li, Three on Three: Universal and High-Affinity Molecular Recognition of the Symmetric Homotrimeric Spike Protein of SARS-CoV-2 with a Symmetric Homotrimeric Aptamer, J. Am. Chem. Soc. 144 (2022) 23465-23473. https://doi.org/10.1021/jacs.2c09870.
[49] Z. Zhang, R. Pandey, J. Li, J. Gu, D. White, H. D. Stacey, J. C. Ang, C. Steinberg, A. Capretta, C. D. M. Filipe, K. Mossman, C. Balion, M. S. Miller, B. J. Salena, D. Yamamura, L. Soleymani, J. D. Brennan, Y. Li, High-Affinity Dimeric Aptamers Enable the Rapid Electrochemical Detection of Wild-Type and B.1.1.7 SARS-CoV-2 in Unprocessed Saliva, Angew. Chemic Int. Ed. 60 (2021) 24266-24274. https://doi.org/10.1002/anic.202110819.
[50] M. L. Walker, D. J. Vanderah, K. A. Rubinson, In-situ characterization of self-assembled monolayers of water-soluble oligo (ethylene oxide) compounds, Colloids Surfaces B Biointerfaces 82 (2011) 450-455. https://doi.org/10.1016/j.colsurfb.2010.09.029.
[51] F. Mo, Z. Chen, G. Liang, D. Wang, Y. Zhao, H. Li, B. Dong, C. Zhi, Zwitterionic Sulfobetaine Hydrogel Electrolyte Building Separated Positive/Negative Ion Migration Channels for Aqueous Zn-MnO2 Batteries with Superior Rate Capabilities, Adv. Energy Mater. 10 (2020). https://doi.org/10.1002/aenm.202000035.
[52] L. Lorencova, V. Gajdosova, S. Hroncekova, T. Bertok, M. Jerigova, D. Velic, P. Sobolciak, I. Krupa, P. Kasak, J. Tkac, Electrochemical Investigation of Interfacial Properties of Ti3C2Tx MXene Modified by Aryldiazonium Betaine Derivatives, Front. Chem. 8 (2020). https://doi.org/10.3389/fchem.2020.00553.
[53] R. Pandey, Y. Lu, E. Osman, S. Saxena, Z. Zhang, S. Qian, A. Pollinzi, M. Smieja, Y. Li, L. Soleymani, T. Hoare, DNAzyme-Immobilizing Microgel Magnetic Beads Enable Rapid, Specific, Culture-Free, and Wash-Free Electrochemical Quantification of Bacteria in Untreated Urine, ACS Sensors 7 (2022) 985-994. https://doi.org/10.1021/acssensors.1c02440.
[54] R. Pandey, D. Chang, M. Smieja, T. Hoare, Y. Li, L. Soleymani, Integrating programmable DNAzymes with electrical readout for rapid and culture-free bacterial detection using a handheld platform, Nat. Chem. 13 (2021) 895-901. https://doi.org/10.1038/s41557-021-00718-x.
[55] A. Victorious, Z. Zhang, D. Chang, R. Maclachlan, R. Pandey, J. Xia, J. Gu, T. Hoare, L. Soleymani, Y. Li, A DNA Barcode-Based Aptasensor Enables Rapid Testing of Porcine Epidemic Diarrhea Viruses in Swine Saliva Using Electrochemical Readout, Angew. Chemic Int. Ed. 61 (2022). https://doi.org/10.1002/anie.202204252.
[56] S. M. Traynor, G. A. Wang, R. Pandey, F. Li, L. Soleymani, Dynamic Bio-Barcode Assay Enables Electrochemical Detection of a Cancer Biomarker in Undiluted Human Plasma: A Sample-In-Answer-Out Approach, Angew. Chemie 132 (2020) 22806-22811. https://doi.org/10.1002/ange.202009664.
[57] Z. Zhang, B. R. Adhikari, P. Sen, L. Soleymani, Y. Li, Functional nucleic acid-based biosensors for virus detection, Adv. Agrochem 2 (2023) 246-257. https://doi.org/10.1016/j.aac.2023.07.006.
[58] L. Zhao, Z. Ma, Facile synthesis of polyaniline-polythionine redox hydrogel: Conductive, antifouling and enzyme-linked material for ultrasensitive label-free amperometric immunosensor toward carcinoma antigen-125, Anal. Chim. Acta 997 (2018) 60-66. https://doi.org/10.1016/j.aca.2017.10.017.
[59] Q. Du, W. Wang, X. Zeng, X. Luo, Antifouling zwitterionic peptide hydrogel based electrochemical biosensor for reliable detection of prostate specific antigen in human serum, Anal. Chim. Acta 1239 (2023) 340674. https://doi.org/10.1016/j.aca.2022.340674.
[60] X. Yang, M. Chen, Z. Zhang, Y. Li, P. Wang, X. Luo, S. Lv, Alpha-aminoisobutyric acid incorporated peptides for the construction of electrochemical biosensors with high stability and low fouling in serum, Anal. Chim. Acta 1238 (2023) 340646. https://doi.org/10.1016/j.aca.2022.340646.
[61] S. Zhao, Y. Zhang, Z. Xu, H. Wang, L. Xu, Y. Wu, X. Zeng, X. Luo, A low-fouling electrochemical biosensor for biomarker detection in serum based on designed a/B-peptides with anti-enzymolysis and antifouling capabilities, Anal. Chim. Acta 1263 (2023) 341244. https://doi.org/10.1016/j.aca.2023.341244.
[62] J. Wang, N. Hui, Zwitterionic poly(carboxybetaine) functionalized conducting polymer polyaniline nanowires for the electrochemical detection of carcinoembryonic antigen in undiluted blood serum, Bioelectrochemistry 125 (2019) 90-96. https://doi.org/10.1016/j.bioclechem.2018.09.006.
[63] T. Zhang, Z. Xu, H. Xu, Y. Gu, Y. Xing, X. Yan, H. Liu, N. Lu, Y. Song, S. Zhang, Z. Zhang, M. Yang, Catechol and zwitterion-bifunctionalized poly(ethylene glycol) based ultrasensitive antifouling electrochemical aptasensor for the quantification of adenosine triphosphate in biological media, Sensors Actuators, B Chem. 288 (2019) 469-475. https://doi.org/10.1016/j.snb.2019.03.027.
[64] T. Kilic, I. Gessner, Y. K. Cho, N. Jeong, J. Quintana, R. Weissleder, H. Lee, Zwitterionic Polymer Electroplating Facilitates the Preparation of Electrode Surfaces for Biosensing, Adv. Mater. 34 (2022) 2107892. https://doi.org/10.1002/adma.202107892.
[65] G. Wang, Q. Xu, L. Liu, X. Su, J. Lin, G. Xu, X. Luo, Mixed Self-Assembly of Polyethylene Glycol and Aptamer on Polydopamine Surface for Highly Sensitive and Low-Fouling Detection of Adenosine Triphosphate in Complex Media, ACS Appl. Mater. Interfaces 9 (2017) 31153-31160. https://doi.org/10.1021/acsami.7b09529.

### Example 2

### Results and Discussions

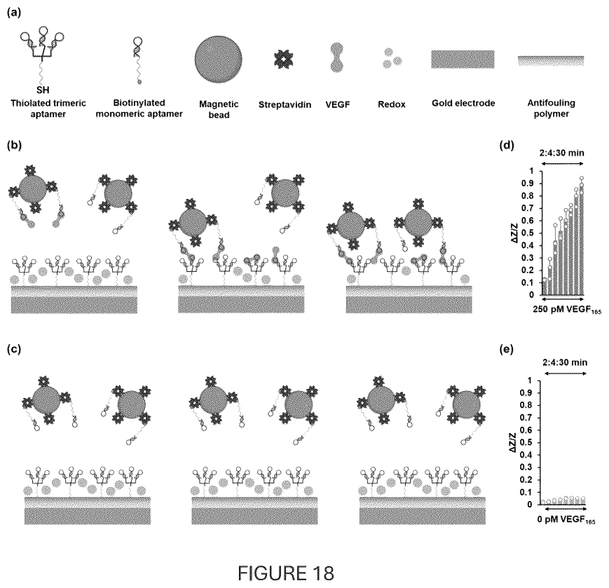

We aimed to create an electrochemical assay capable of directly monitoring a wide range of aptamer-target interactions on electrode surfaces in real time. Previously, we developed the Real Time Multimeric Aptamer (RT-MAp) Assay to address this goal, which translates aptamer-virus binding into changes in electrochemical impedance.^{[1]} The challenge with this assay was that even though it is highly effective in detecting large targets, such as viruses that cause a significant change in electrochemical impedance, ^{[1]} it can be difficult to detect aptamer targets like proteins that are smaller in size.^{[2]} In response, we developed a Real Time Magnetic beads Multimeric Aptamer (RT-MagMAp) assay on Antifouling polymer modified electrode surface (Antifouling RT-MagMAp) to enable highly sensitive detection of a wide range of targets including proteins (FIG. 18a-c).

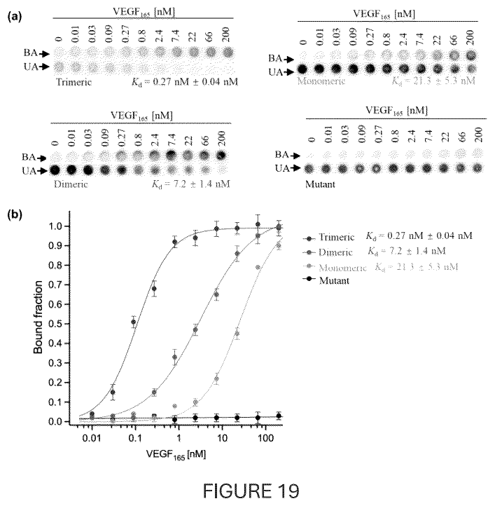

The Antifouling RT-MagMAp Assay is designed to form a sandwich structure on the electrode surface only in the presence of the target (FIG. 18b), which leads to a measurable change in electrochemical impedance. More specifically, the target is sandwiched between two VEGF₁₆₅ aptamers (FIG. 18b), with trimeric aptamers (FIG. 18a) (FIG. 19) immobilized on the electrode surface and biotinylated monomeric aptamers (FIG. 18a) (FIG. 19) immobilized on commercially available streptavidin modified magnetic beads (FIG. 18a-c). The electrodes are modified with a thiol-terminated anti-biofouling DMAPS polymer that can bind the electrode through Au-S interactions and thiolated trimeric aptamers to the DMAPS polymer via S-S bond (FIG. 18a-c). The polymer coating is designed to produce low interfacial energy and hydrophilicity for inhibiting nonspecific binding. (3) The magnetic beads play two key roles in the assay. They are used to: 1) extract target molecules from sample solutions and 2) enhance the impedance change generated on the electrode surfaces in response to target binding to enable the detection of protein targets that would not otherwise generate a sufficient signal change. Single frequency impedance measurement is used to characterize the electrode surfaces in real time using electrochemical kinetic profiling. The formation of the sandwich structure in the presence of the target inhibits the access of the redox couple ([Fc(CN)₆]⁴⁻/[Fe(CN)₆]³⁻) to the electrode surface (FIG. 18b), increasing the electrochemical impedance compared to the blank case that lacks the sandwich structure (FIG. 18c).

We evaluated the assay by performing single frequency impedance (Z) measurements every 2 minutes over a 30-minute period and calculating the rate of change of impedance (ΔZ/Z) from the first point of measurements (t=0), and plotting ΔZ/Z every 4 minutes (FIG. 18d-e). In the presence of 250 pM VEGF₁₆₅ spiked into 20% plasma, the ΔZ/Z signal progressively increases over 30 minutes, indicating the capture of additional magnetic beads and the formation of sandwich complexes on the trimeric aptamer-functionalized anti-biofouling electrodes (FIG. 18d). In contrast, in the absence of VEGF₁₆₅, no magnetic bead capture occurs, and only a negligible increase in the ΔZ/Z signal is observed (FIG. 18e). This minimal change (FIG. 18e) can be attributed to either the photochemical degradation of the redox probe, leading to change in the ionic strength of the electrolyte,^{[4]} or minor adsorption of plasma components on the DMAPS polymer-coated electrode.^{[3]}

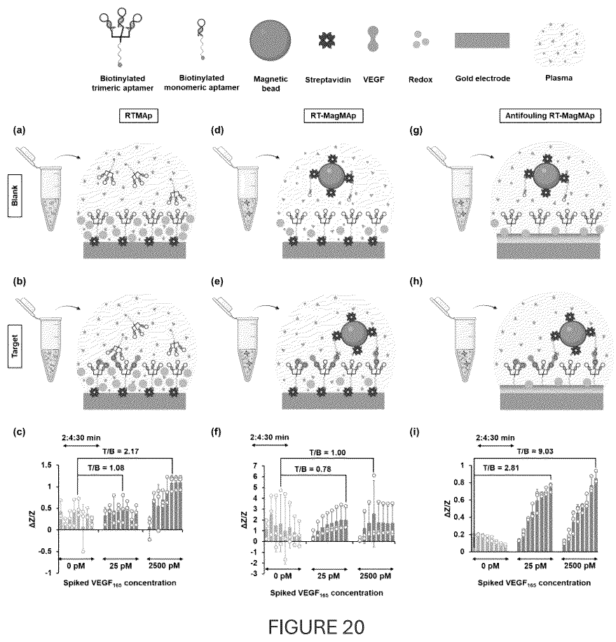

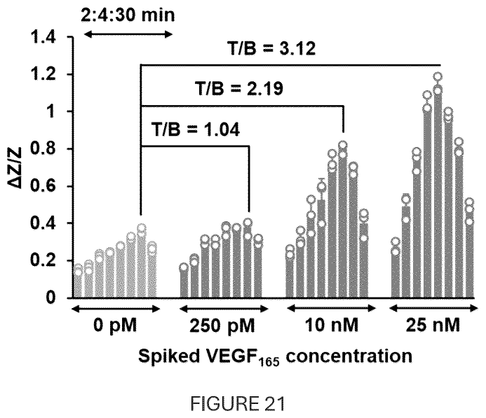

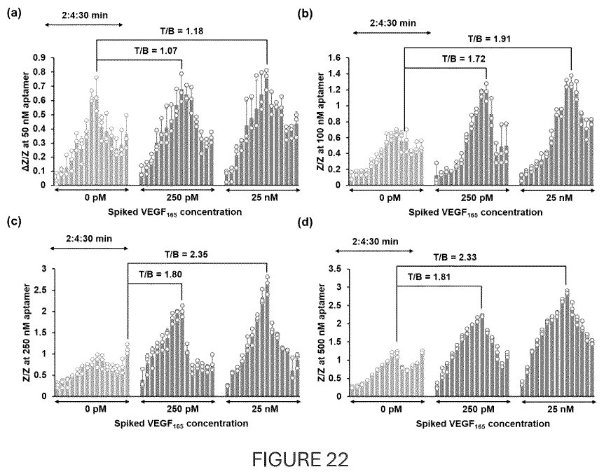

To demonstrate the need for the magnetic beads for target extraction and signal enhancement, we compared the performance RT-MAp assay which we had used in our previous study^{[1]} with RT-MagMAp assay with and without magnetic purification, without DMAPS coating (FIG. 20) in detecting VEGF₁₆₅ in diluted plasma. To start with we recorded the fold change in impedance (ΔZ/Z) for RT-MAp assay (FIG. 20a) for 0 to 25 nM VEGF₁₆₅ spiked in binding buffer (FIGS. 21) and 20% plasma (FIG. 20b) using optimal concentration of biotinylated trimeric aptamers (FIG. 22). Despite positive results for SARS-CoV-2 detection with ˜30 spike proteins per virus, the RT-MAp assay (FIG. 20a) showed poor resolution and very fluctuating trend (T−B=0.55 for 250 pM VEGF₁₆₅ and T−B=0.3 for 2500 pM VEGF₁₆₅) with high ΔZ/Zₘₐₓ deviation of 12-50% even for 250 pM VEGF₁₆₅ in 20% plasma diluted in binding buffer (FIG. 20b).

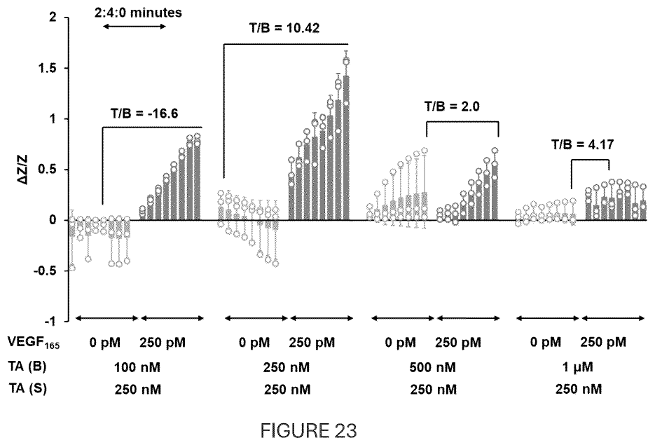

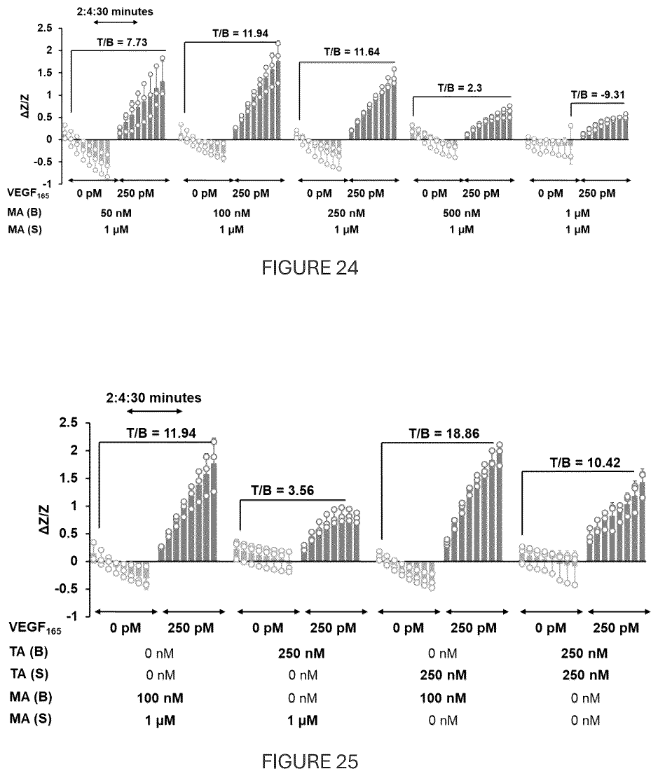

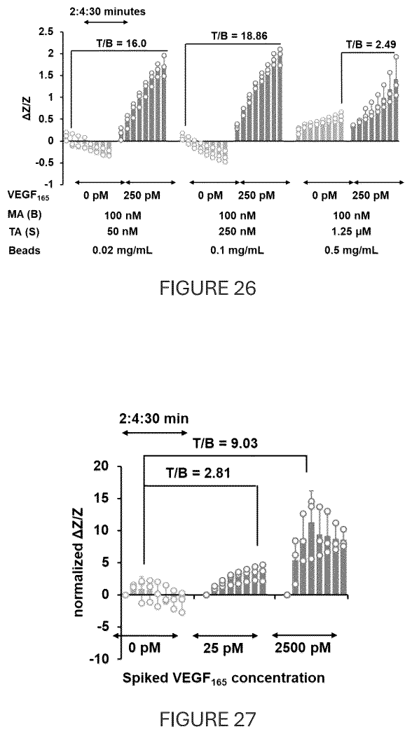

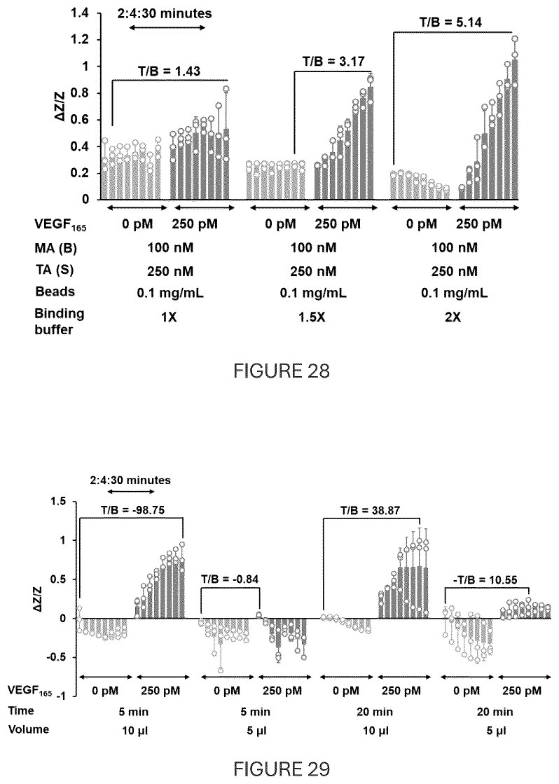

With the intention of improving resolution in plasma, we employed RT-MagMAp (FIG. 20c), for which we introduced mixture of the reagent mix and spiked plasma directly onto the chip, initiating kinetics immediately. At optimal conditions (FIGS. 23-26) RT-MagMAp without magnetic purification did not produce much resolution as anticipated (FIG. 27) as evident from the T−B=−0.55 obtained for 250 pM VEGF₁₆₅ and T−B=2E−4 for 25 nM VEGF₁₆₅ along with a high deviation of 45-123% for 250 pM VEGF₁₆₅. Although obtained signals were very variable, ΔZ/Z signals at each time point normalized with respect to 2-minute ΔZ/Z signals (FIG. 20d) showed good resolution not only between blank and target but between different concentrations, as observed from T−B=2.25 for 250 pM VEGF₁₆₅ and T−B=10.03 for 25 nM VEGF₁₆₅. However, a high deviation is still obtained for the signals (45-129% for VEGF₁₆₅) (FIG. 20d). and RT-MagMAp made analysis and interpretation complex. Purified RT-MagMAp introduced pre magnetically purified beads suspended in reagent mixture on the chip for recording kinetics (FIG. 20e). Although variability was significantly improved (4-27% for 250 pM VEGF₁₆₅), poor resolution was still observed (T−B=0.17 for 250 pM VEGF₁₆₅) which was resolved by using 2× binding buffer (FIG. 28) under optimal conditions (FIG. 29) as observed from T−B=0.85 for 250 pM VEGF₁₆₅, which is in agreement with previous studies.^{[28,29]}

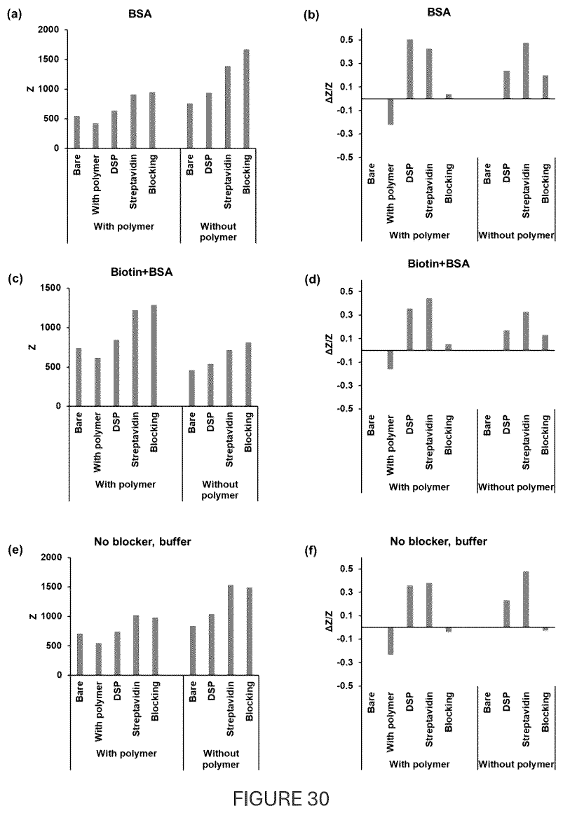

Next, in order to justify the utility of DMAPS polymer coat, we compared the performance of RT-MagMAp and Purified RT-MagMAp, which incorporated magnetic beads but without polymer coat with Antifouling RT-MagMAp which incorporated both magnetic beads and polymer coat. It is to be noted here that unlike RT-MAP, RT-MagMAp, and Purified RT-MagMAp,which used streptavidin-biotin chemistry, Antifouling RT-MagMAp used thiolated aptamers which bound to thiol terminal of DMAPS polymer coat. This is because streptavidin modification required DSP linkers soluble in DMSO; DMAPS also soluble in DMSO posed a risk to polymer stability. Investigation showed that BSA is not required for Antifouling RT-MagMAp due to the polymer coating on the electrodes (FIG. 30). This simplifies the assay by removing BSA, which introduced competition in the binding mixture of the other assays.

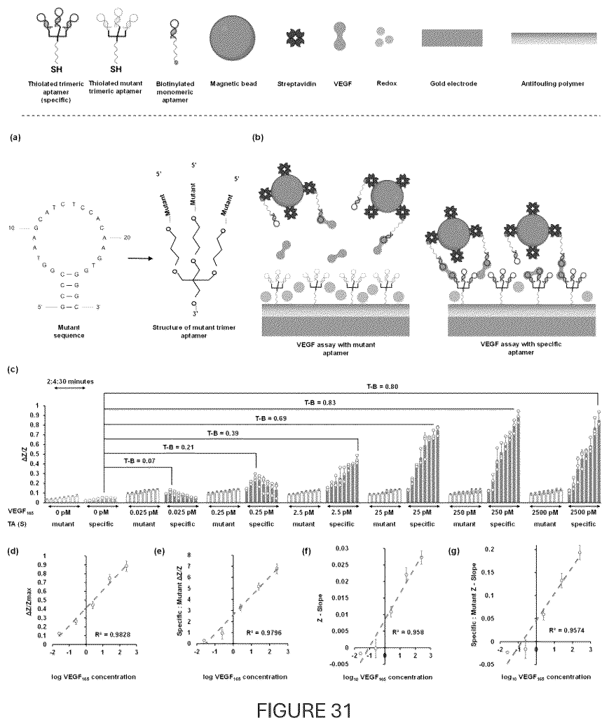

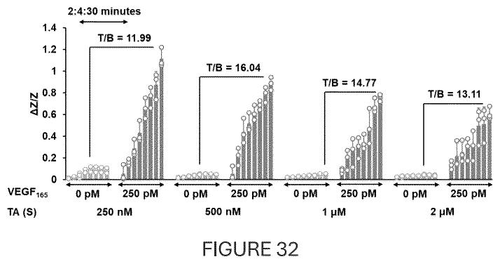

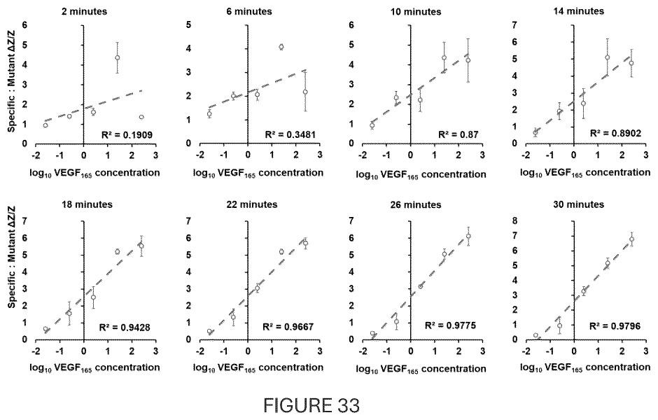

Under optimal conditions (FIG. 31), ΔZ/Z signals were recorded for Antifouling RT-MagMAp for 0 to 2500 pM VEGF₁₆₅ in 20% plasma using the VEGF₁₆₅ specific trimeric aptamer and a mutant control aptamer which exhibited minimal interaction with VEGF₁₆₅ (FIG. 32). The control experiments were designed such that the surface bound specific aptamers are replaced with the same concentration of mutant aptamers. A poor resolution was obtained below 0.25 pM VEGF₁₆₅ using the specific aptamer alone (T−B=0.07 for 0.025 pM VEGF₁₆₅ and T−B=0.21 for 0.25 pM VEGF₁₆₅. However considerable resolution was obtained for concentrations above 0.25 pM with signals saturating at 250 pM (T−B=0.83 for 250 pM and T−B=0.8 for 2500 pM VEGF₁₆₅. We then performed control baseline cancellation by calculating the ratio of specific ΔZ/Z signal to mutant ΔZ/Z signal (Specific: Mutant ΔZ/Z ratio) at each time point as described in equation (1) (FIG. 33b).

$$Specific : Mutant {\frac{Δ Z}{Z}}_{i} = \frac{Specific {\frac{Δ Z}{Z}}_{i}}{Average mutant \frac{Δ Z}{Z}} \qquad ( 1 )$$

Considering specific: mutant ΔZ/Z ratio, despite saturation at 250 pM VEGF₁₆₅ This led to a clear improvement in resolution as can be observed from the T−B values (FIG. 33b), indicating the importance of mutant aptamer integration as a parallel control. We further evaluated the specificity of the obtained signal by testing it against viral protein biomarkers (COVID Spike protein, Influenza A H1N1, and H3N2) as well as a typical cancer biomarker (Prostate Specific Antigen). All non-specific signals were below the threshold with respect to specific signal of 0 nM VEGF₁₆₅ (black dotted line in FIG. 33c, evaluated according to equation 2) and threshold with respect to mutant signal corresponding to the highest analyzed concentration of VEGF₁₆₅ (2.5 nM) (black solid line in FIG. 33c, evaluated according to equation 3), demonstrating the assay's specificity.

$$Threshold wrt 0 nM {VEGF}_{165} = {\frac{Δ Z}{Z}}_{specific} + 3 \times standard deviation of {\frac{Δ Z}{Z}}_{specific} \qquad ( 2 )$$

$$Threshold wrt 2.5 nM {VEGF}_{165} = {\frac{Δ Z}{Z}}_{mutant} - 3 \times standard deviation of {\frac{Δ Z}{Z}}_{mutant} \qquad ( 3 )$$

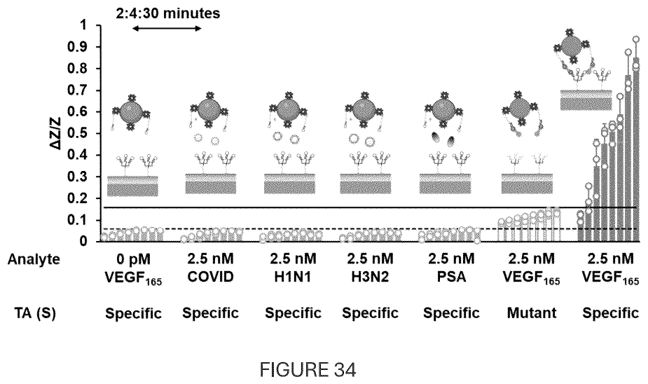

Next, we aimed to utilize the signals, thus recorded and analyzed to calibrate the sensor. First, we used the maximum ΔZ/Z signal (ΔZ/Zₘₐₓ) obtained using only the VEGF₁₆₅ specific aptamer to calibrate the sensor. We conducted a log-linear fit (R²=0.9828) to the ΔZ/Zₘₐₓ signals across 0 to 250 pM VEGF₁₆₅ (FIG. 34a) which yielded an LOD of 32 fM using Antifouling RT-MagMAp. This calibration method using ΔZ/Zₘₐₓ was first investigated for its simplicity and effectiveness; however, it lacked a mechanism to account for sample-to-sample variability.

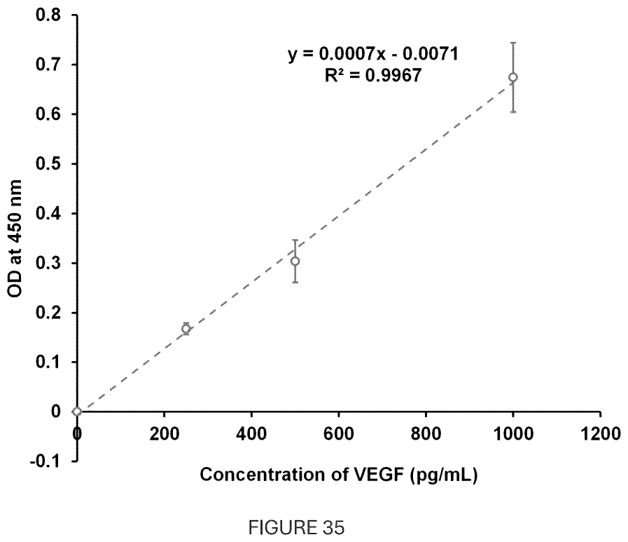

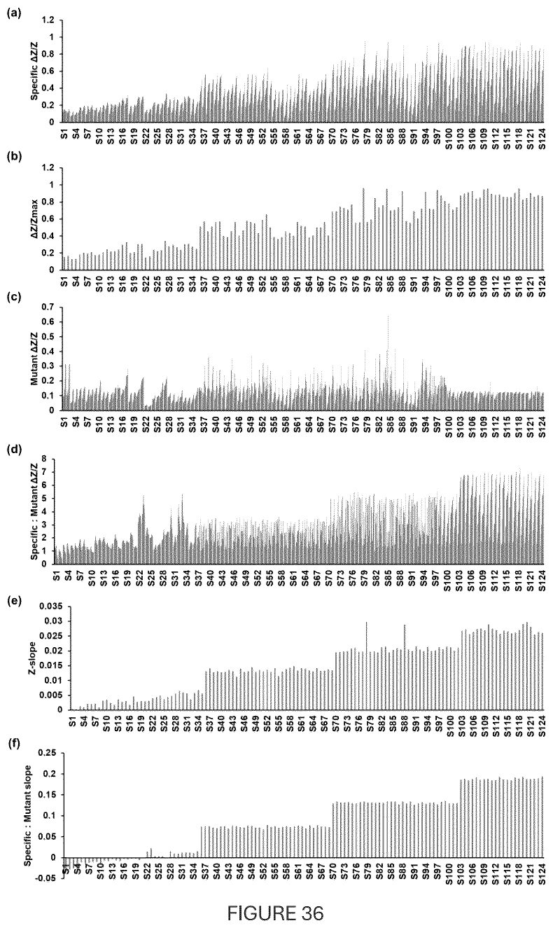

To address the limitation of the ΔZ/Zₘₐₓ based calibration method and incorporate the improved sensitivity previously obtained using specific: mutant ΔZ/Z signal (FIG. 34b), we conducted log-linear fits at each time point and selected the one with the best fit (R²=0.9796) between 0.025 pM and 250 pM, which occurred at 30 minutes (FIGS. 35 and 36). This method yielded a limit of detection of 106 fM (FIG. 34b), which was 3.3 times poorer than the detection limit obtained with the first method (FIG. 34a). For the third calibration method, we focused on the increase in specific ΔZ/Z signals over 28 minutes, starting from the ΔZ/Z signal at 2 minutes (Z-slope), using equation (4) (FIG. 34c).

$$Z - slope = \frac{Specific {\frac{Δ Z}{Z}}_{t = 30} - Specific {\frac{Δ Z}{Z}}_{t = 2}}{28} \qquad ( 3 )$$

The log linear fit between 0.025 pM and 250 pM VEGF₁₆₅ (R²=0.958) resulted in a limit of detection of 183 fM (FIG. 33f), which was 5.7 times poorer than that obtained from ΔZ/Zₘₐₓ based calibration and 1.7 times poorer than calibration considering mutant aptamer signal. For the final calibration we incorporated mutant aptamer and calculated the increase in Specific: Mutant ΔZ/Z signal ratio over 28 minutes, starting from the Specific: Mutant ΔZ/Z signal at 2 minutes (Specific: Mutant Z-slope), using equation (4). The log linear fit (R²=0.9574) yielded a limit of detection of 354 fM (FIG. 34d), which is 1.9 times poorer than Z-Slope without mutant.

$$Specific : Mutant Z - slope = \frac{Specific : Mutant {\frac{Δ Z}{Z}}_{t = 30} - Specific : Mutant {\frac{Δ Z}{Z}}_{t = 2}}{28} \qquad ( 4 )$$

Notably the four calibration methods yielded progressively poorer limits of detection (FIG. 34) but had their own advantages. While ΔZ/Zₘₐₓ based calibration did not consider any control baseline, specific: mutant ΔZ/Z ratio considered mutant aptamer signals for incorporating false signals from the plasma itself. Z-slope based calibration method further cancelled the variability in the initial signal, leading to greater reliability, while Specific: Mutant Z-Slope did an initial signal cancellation along with incorporating of the mutant control baseline.

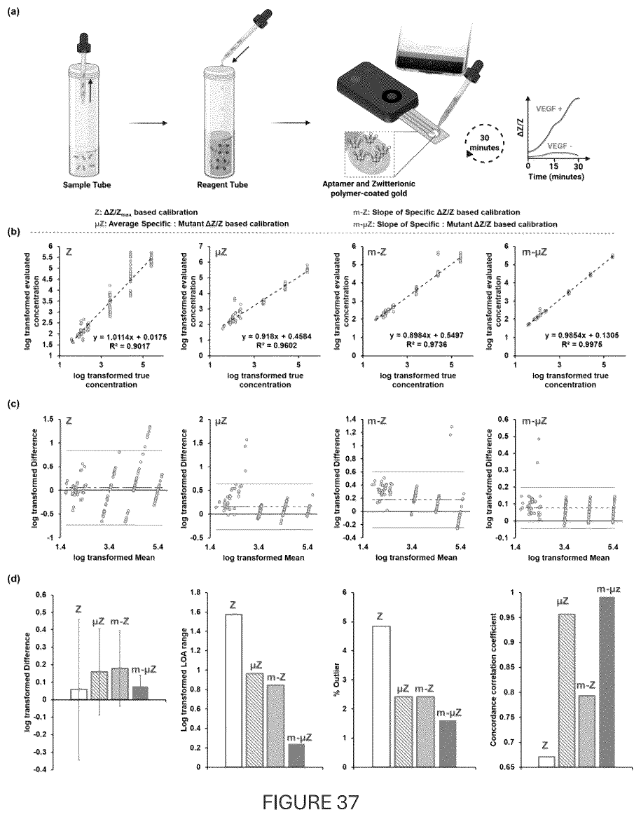

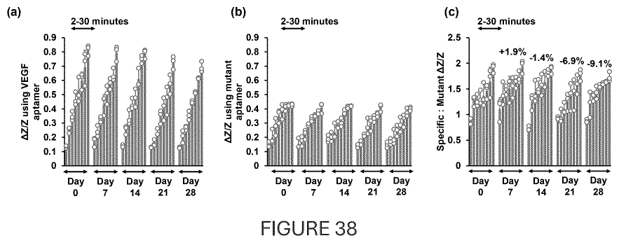

In order to determine the calibration method most effective for quantifying trace VEGF₁₆₅ in multiple plasma samples, we prepared a set of plasma samples with varying spiked VEGF₁₆₅ concentrations. We quantified 12 plasma samples (including the one used to establish the limit of detection) using ELISA, using the latter as the calibration sample for ELISA (FIG. 37). The total VEGF₁₆₅ concentration in each evaluated sample was therefore the sum of the ELISA-quantified concentration and the spiked VEGF₁₆₅ concentration. We prepared 124 samples and then assessed the VEGF₁₆₅ concentration in each using the four calibration methods described in FIG. 34 (FIG. 35). The results were then compared with the total VEGF₁₆₅ concentration present in each sample.

The evaluated VEGF₁₆₅ concentrations were then plotted against the true VEGF₁₆₅ concentrations to assess their alignment. A linear regression was performed on the log-transformed evaluated and true concentrations to quantify the degree of agreement. The closer the fitted line is to the y=x reference, the stronger the alignment. Among the evaluated models, the Specific: Mutant Z-Slope calibration exhibited the most linear fit (R²=0.9975) and a slope closest to 1. It is to be noted here that although the ΔZ/Zₘₐₓ calibration also showed a slope near 1, its poor linearity (R²=0.9017) disqualified it as the optimally aligned model (FIG. 36a). To further evaluate agreement, log-transformed Bland-Altman plots^{[5-7]} were employed (FIG. 36b). The proximity of the mean difference (dotted red line, equation (5)) to zero (black line) indicated improved quantification accuracy. The standard deviation of the differences and the spread of the limits of agreement (LOA, top and bottom blue lines, equations (6-8)), calculated with 95% confidence intervals (CI=1.96), determined the consistency of the model. Outliers falling beyond these limits were observed to be randomly distributed (FIG. 36b) across all four calibration methods, suggesting experimental variability rather than systematic bias. A smaller and less variant mean difference, a more condensed LOA range, and fewer outliers (FIG. 36c) confirmed that the Specific: Mutant Z-Slope method achieved the most precise quantification.

$$Mean Difference = \frac{1}{n} {\sum }_{i = 1}^{n} ( {X}_{i} - {Y}_{i} ) \qquad ( 5 )$$

$$Upper LOA = Mean Difference + 1.96 \times SD of Mean Difference \qquad ( 6 )$$

$$Lower LOA = Mean Difference - 1.96 \times SD of Mean Difference \qquad ( 7 )$$

$$LOA range = Upper LOA - Lower LOA \qquad ( 8 )$$

This was further supported by the highest obtained Pearson's correlation coefficient (PCC=0.992, equation (9)^{[6,7]}) (FIG. 36c) obtained by Specific: Mutant Z-Slope calibration method.

$$PCC = \frac{\sum ( {X}_{i} - mean X ) ( {Y}_{i} - mean Y )}{\sqrt{\sum {( {X}_{i} - mean X )}^{2}} \sqrt{{( {Y}_{i} - mean Y )}^{2}}} \qquad ( 9 )$$

The combined PCC and Bland-Altman analysis^{[7]} (FIG. 36) highlighted the advantages of incorporating a mutant aptamer over solely relying on maximum ΔZ/Z signals. It further emphasized the importance of compensating for initial non-specific signals immediately after sample introduction by taking the Slope. This finding is particularly valuable to design robust clinically applicable systems with self-calibration.

The calibrated sensor exhibited excellent stability over a period of 28 days, indicating a good shelf life when stored in a vacuum-sealed pouch at 4° C. after air drying. Additionally, the 10 μL of aptamer-functionalized beads (as described in the Methods section) were also stored at 4° C. (FIG. 37). The Specific: Mutant ΔZ/Z remained nearly unchanged until Day 14, with only a slight signal reduction of 6.9% observed on Day 21 and a 9.1% reduction by Day 28, compared to the signal measured on Day 0 (FIG. 37).

### Conclusion

In this study, we developed the Antifouling Real-Time Magnetic Multimeric Aptamer (Antifouling RT-MagMAp) assay, a novel electrochemical biosensing platform that achieves real-time detection of low-abundance protein biomarkers such as VEGF₁₆₅ directly in untreated diluted human plasma.

Beyond its analytical performance, the Antifouling RT-MagMAp assay represents a meaningful step toward next-generation diagnostics. By applying an extensive analysis of different calibration methods, we identified that monitoring the evolution of specific aptamer's impedance signal normalized against the mutant aptamer's impedance response provided the most consistent, reproducible and reliable quantification method across different patient samples and replicates. By combining magnetic bead-labelled monomeric aptamers with a sandwich hybridization strategy and integrating an antifouling DMAPS polymer layer, the assay overcomes key limitations of conventional aptamer sensors, particularly signal drift, nonspecific adsorption, and challenges in kinetic resolution for small targets through optimized calibration. This advancement allows reliable, time-resolved impedance readouts with excellent sensitivity, dynamic range, and reproducibility, even in complex biological matrices.

### Materials and Methods

### Materials

All chemicals and reagents, including isopropyl alcohol (IPA), 98% sulfuric acid (H₂SO₄), 4-(2-hydroxyethyl)-1-piperazineethanesulfonic acid (HEPES), sodium chloride, magnesium chloride, Tween-20, bovine serum albumin, K₃[Fe(CN)₆], K₄[Fe(CN)₆], dithiobis[succinimidylpropionate] (DSP), dimethyl sulfoxide (DMSO), and tris(2-carboxyethyl)phosphine (TCEP), were purchased from Sigma-Aldrich (Oakville, Canada) and used without further purification. Streptavidin coated magnetic dynabeads were purchased from Thermofischer Scientific. Electrochemical tests were carried out using screen-printed gold electrodes with silver reference and gold auxiliary electrodes from Palmsens. Ultrapure water (Milli-Q System, Millipore) was used to prepare all aqueous solutions.

### ELISA

Human VEGF₁₆₅ ELISA Colorimetric Kit was procured from Abcam and used for plasma sample quantification.

### Methods

### Electrode Cleaning and Quality Control

The screen-printed electrodes were cleaned using IPA and DI water. The working electrode was activated through 15 cyclic voltammetry scans in 0.5 M H₂SO₄, spanning from 0 V to 1.5 V at a scan rate of 10 mV/s. After activation, the chip was rinsed with DI water. Next, two bare cyclic voltammetry scans were performed using a 1× readout buffer (1× phosphate-buffered saline (PBS), 50 mM KCl, 2 mM ferrocyanide (K₃[Fe(CN)₆]), and 2 mM ferricyanide (K₄[Fe(CN)₆])). These scans were conducted immediately after cleaning, with the second scan used for analysis. Chips with redox currents less than 40 μA were discarded.

### Electrode Modification

For RT-MAp, RT-MagMAp and Purified RT-MagMAp assay (FIG. 21): To prepare the DSP, 2 mg of DSP was mixed with 500 μL of DMSO by vortexing. For DSP reduction, 50 μL of the DSP-DMSO solution was combined with 450 μL of TCEP dissolved in DMSO and thoroughly mixed. The mixture was incubated for at least 1 hour. Following this, 3.5 μL of the TCEP-reduced DSP was deposited onto the working electrode and incubated for 2 hours. The chip was then washed with DMSO and subsequently with DI water. Next, 5 μL of streptavidin, diluted to a concentration of 0.5 μM in 1× PBS, was added to the working electrode and allowed to incubate overnight at 4° C. The chips were washed in 1× PBS and incubated with 5 μL of 250 nM biotinylated trimeric VEGF₁₆₅ aptamer for 30 minutes. The chips were then washed with 1× binding buffer (50 mM HEPES, pH 7.4, 150 mM NaCl, 6 mM KCl, 2.5 mM MgCl₂, 2.5 mM CaCl₂) and stored for further use.

For Antifouling RT-MagMAp assay (FIG. 21a): A multi-functional zwitterionic polymer DMAPS90-MAA-SH10 was prepared using free radical copolymerization as described in our previous work.^{[8]} The cleaned chips, with all areas except the working electrode masked, were dip-coated for 24 hours in this freshly-made polymer solution. Briefly, this polymer solution was prepared by dissolving 60 mg of polymer in 2 mL of autoclaved DI water, mixed with 0.19 M TCEP to ensure the presence of free thiols, and then dialyzing out the excess TCEP using a 3.5 kDa membrane for 2 hours against 10 mM NaCl under stirring at 80 RPM. After dialysis, the product was diluted to a concentration of 4 mg mL⁻¹ using DI water and 1.5 mL of 10× PBS, resulting in a final volume of 15 mL in 1× PBS. After 24 hours of dip-coating, the electrodes were removed from the polymer solution, gently dried with a Kimwipe, and the mask was removed. Then the polymer coated working electrode was air dried and taken for aptamer deposition. In order to ensure consistency in polymer coating batches of 12 electrodes were coated in 7.5 ml of polymer solution. 5 μM of thiolated trimeric VEGF₁₆₅ aptamer was reduced for 2 hours at RT in dark by 50 μM of TECP in water followed by dilution to 500 nM of reduced thiolated trimeric VEGF₁₆₅ aptamer using 1× binding buffer. Then 5 μL of the reduced aptamer was deposited on the thiol terminated polymer modified air-dried electrodes overnight at RT in dark. The next day, the electrodes were washed with water and stored for further use.

### Magnetic Bead Functionalization and Purification

For RT-MagMAp, Purified RT-MagMAp and Antifouling RT-MagMAp assay (FIG. 21, FIG. 18): The as-obtained streptavidin coated magnetic beads were washed by mixing 5 μL of 10 mg mL⁻¹ magnetic beads with 20 μL of binding buffer containing 0.01% TWEEN-20 in an Eppendorf tube. The solution was mixed thoroughly with a pipette and then purified for 10 minutes on an external magnet holder. After magnetic separation, 18 μL of the supernatant was removed, and 18 μL of binding buffer containing 0.01% TWEEN-20 was added to the remaining solution. This washing process was repeated twice. After the final wash, 15 μL of binding buffer containing 0.01% TWEEN-20 was added to the remaining 5 μL of washed beads.

In order to functionalize the streptavidin-coated beads with biotinylated monomeric VEGF₁₆₅ aptamer, the 20 μL solution (5 μL of washed beads+15 μL of binding buffer containing 0.01% TWEEN-20) was separated into two Eppendorf tubes. Then, 10 μL of a 10 μM monomeric aptamer was added in each tube. The solution was incubated for 30 minutes, with mixing every five minutes using a pipette. For purification, the tubes were placed on the magnet holder for 15 minutes to magnetically separate out the aptamer functionalized beads from excess aptamers. After magnetic separation, 17 μL of the supernatant (expected to contain unbound aptamers) was removed, and 27 μL of 1× binding buffer was added to the remaining solution. Then 5 μL aliquots of the resultant suspension was created and stored in 4° C. to be used for each chip during sensing.

### Spiked Sample Testing

For RT-MAp (FIG. 21a): A 50 μL one-pot sensing blank (without VEGF₁₆₅) and target (with VEGF₁₆₅) mix was prepared. Blank was prepared by mixing 10 μL plasma sample, 5 μL of 0.1% BSA, 5 μL of 10× binding buffer, 5 μL of autoclaved water and 25 μL of 2× readout buffer. Target was prepared by mixing 10 μL plasma sample pre-spiked with 5 μL VEGF₁₆₅, 5 μL of 0.1% BSA, 5 μL of 10× binding buffer and 25 μL of 2× readout buffer. In this assay version, biotinylated BSA, which was used in the original RT-MAp assay,^{[1]} was excluded to prevent the beads from binding non-specifically to the biotin. This could occur if the biotinylated BSA adsorbs onto the chip with the BSA terminal on the gold electrode. The resulting mixture was then applied to the streptavidin and biotinylated trimeric VEGF₁₆₅ aptamer modified chips, covering all three electrodes (working, reference, and counter), and single-frequency impedance recording was initiated immediately.

For RT-MagMAp (FIG. 21b): A 50 μL one-pot sensing blank (without VEGF₁₆₅) and target (with VEGF₁₆₅) mix was prepared. Blank was prepared by mixing 10 μL plasma sample, 5 μL of monomeric aptamer functionalized and pre-purified magnetic beads, 5 μL of 10× binding buffer, 0.5 μL of 1% BSA, 5 μL of autoclaved water and 25 μL of 2× readout buffer. Target was prepared by mixing 10 μL plasma sample pre-spiked with 2.5 μL of 20× VEGF₁₆₅, 5 μL of monomeric aptamer functionalized and pre-purified magnetic beads, 5 μL of 10× binding buffer, 0.5 μL of 1% BSA, 2 μL of autoclaved water and 25 μL of 2× readout buffer. The resulting mixture was then applied to the streptavidin and biotinylated trimeric VEGF₁₆₅ aptamer modified chips, covering all three electrodes (working, reference, and counter), and single-frequency impedance recording was initiated immediately.

For Purified RT-MagMAp (FIG. 21c): A 50 μL one-pot sensing blank (without VEGF₁₆₅) and target (with VEGF₁₆₅) mix was prepared. The blank set included 10 μL of 10× binding buffer, 10 μL of plasma, 5 μL of conjugated beads and 5 μL of autoclaved water. The target set consisted of 10 μL of 10× binding buffer, 10 μL of plasma pre-spiked with 5 μL of 10× VEGF₁₆₅ and 5 μL of conjugated beads. After mixing the solutions, they were pre-incubated for 5 minutes. The solutions were then placed on a magnet holder for 3 minutes to magnetically separate the beads, and 20 μL of the supernatant was removed. The remaining 10 μL aliquots (both blank and target) were then resuspended in 5 μL binding buffer, 5 μL of 1% BSA, 5 μL autoclaved water and 25 μL 2× readout buffer. The resulting mixture was then applied to the streptavidin and biotinylated trimeric VEGF₁₆₅ aptamer modified chips, covering all three electrodes (working, reference, and counter), and single-frequency impedance recording was initiated immediately.

For Antifouling RT-MagMAp (FIG. 18): A 50 μL one-pot sensing blank (without VEGF₁₆₅) and target (with VEGF₁₆₅) mix was prepared. The blank set included 5 μL of 20× binding buffer, 10 μL of plasma, 5 μL of conjugated beads, 5 μL of autoclaved water and 25 μL of 2× readout buffer. The target set included 5 μL of 20× binding buffer, 10 μL of plasma pre-spiked with 5 μL of 2× VEGF₁₆₅, 5 μL of conjugated beads, and 25 μL of 2× readout buffer. The resulting mixture was then applied to the antifouling polymer and thiolated trimeric VEGF₁₆₅ aptamer modified chips, covering all three electrodes (working, reference, and counter), and single-frequency impedance recording was initiated immediately.

### Plasma VEGF165 Quantification Using ELISA

Human VEGF₁₆₅ ELISA Kit (consisting of VEGF₁₆₅ coated strips, VEGF₁₆₅ standard, standard diluent buffer, biotinylated antibody, biotinylated antibody diluent, HRP (Horseradish Peroxidase) diluent, streptavidin-HRP, wash buffer, TMB (3,3′,5,5′-Tetramethylbenzidine) substrate and stop reagent) obtained from abcam (ab273164) was used for the measurement of VEGF₁₆₅ in the human plasma samples and the protocol provided with the kit was followed. Plasma samples were centrifuged at 1000×g for 30 minutes to remove the particulates and then diluted to 1:2 in standard diluent buffer. VEGF₁₆₅ standards (1000, 500, 250, 0 pg mL⁻¹) were also prepared in diluted plasma. After preparing the reagents as per the kit protocol, 100 μL of standards and plasma samples in triplicates were loaded into the wells, followed by incubation for 2 hours at room temperature. The wells were then washed thrice using 300 μL of 1× washing buffer, and then 50 μL of biotinylated antibody was added to the wells for 1 hour at room temperature followed by three times washing with washing buffer. Afterwards, 100 μL of streptavidin-HRP was added for 30 minutes at room temperature and washed thrice before adding 100 μL of TMB substrate for 10-15 minutes in dark. In the end, 100 μL of stop reagent was added to the wells, and then the absorbance reading was obtained at 450 nm using a Tecan Infinite 200 Pro plate reader. A calibration curve was obtained from the absorbance readings of the standards, which was then used to calculate the amount of VEGF₁₆₅ present in plasma samples.

### References

[1] P. Sen, Z. Zhang, S. Sakib, J. Gu, W. Li, B. R. Adhikari, A. Motsenyat, J. L'Heureux-Hache, J. C. Ang, G. Panesar, B. J. Salena, D. Yamamura, M. S. Miller, L. Soleymani, Y. Li, Angew Chem Int Ed 2024, c202400413.
[2] A. Ch. Lazanas, M. I. Prodromidis, ACS Meas. Sci. Au 2023, 3, 162-193.
[3] S. Saxena, Y. Lu, Z. Zhang, Y. Li, L. Soleymani, T. Hoare, Chemical Engineering Journal 2024, 495, 153522.
[4] S. Vogt, Q. Su, C. Gutiérrez-Sánchez, G. Nöll, Anal. Chem. 2016, 88, 4383-4390.
[5] A. M. Euser, F. W. Dekker, S. Le Cessie, Journal of Clinical Epidemiology 2008, 61, 978-982.
[6] R. Abbasi, M. Imanbekova, S. Wachsmann-Hogiu, Biosensors and Bioelectronics 2024, 254, 116200.
[7] R. Zayat, A. Goetzenich, J.-Y. Lee, H. Kang, S.-H. Jansen-Park, T. Schmitz-Rode, G. Musetti, H. Schnoering, R. Autschbach, N. Hatam, A. Aljalloud, PeerJ 2017, 5, e4132.
[8] S. Saxena, Y. Lu, Z. Zhang, Y. Li, L. Soleymani, T. Hoare, Chemical Engineering Journal 2024, 495, 153522.

While the present invention has been described with reference to examples, it is to be understood that the scope of the claims should not be limited by the embodiments set forth in the examples but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term herein is found to be defined differently in a document incorporated herein by reference, the definition provided herein is to serve as the definition for the term.

What is claimed is:

1. A polymer coating for an electrochemical biosensor comprising one or more polymerizable zwitterionic monomers containing at least one cationic charge and at least one anionic charge at neutral pH and one or more first polymerizable comonomers that contains or can be modified to contain thiol groups.

2. The polymer coating of claim 1, wherein the coating does not require any blocker, spacer, or backfiller to reduce electrode fouling.

3. The polymer coating of claim 1, wherein the thiol groups enable covalent, physical, or affinity binding of a sensing probe.

4. The polymer coating of claim 1, wherein the first polymerizable comonomer contains or can be modified to contain carboxyl groups.

5. The polymer coating of claim 1, further comprising one or more second polymerizable comonomers that enables covalent, physical, or affinity binding of a sensing probe.

6. The polymer coating of claim 5, wherein the second polymerizable comonomer contains or can be modified to contain aldehyde groups.

7. The polymer coating of claim 1, wherein the zwitterionic monomer comprises a sulfobetaine, carboxybetaine, a phosphorylcholine, or a combination thereof.

8. The polymer coating of claim 1, wherein the zwitterionic monomer comprises [2-(methacryloyloxy)ethyl]dimethyl-(3-sulfopropyl)ammonium hydroxide (DMAPS), 2-methacryloyloxyethyl phosphorylcholine (MPC), carboxybetaine methacrylate (CBMA), or combinations thereof.

9. The polymer coating of claim 1, wherein the first comonomer is selected from methacrylic acid, acrylic acid, vinylacetic acid, fumaric acid, and maleic acid.

10. The polymer coating of claim 5, wherein the second comonomer is N-(2,2-dimethoxyethyl) methacrylamide (DMEMA).

11. The polymer coating of claim 1, wherein the coating is attached to a noble metal or noble metal-coated electrode via noble metal-thiol interactions.

12. The polymer coating of claim 11, wherein the noble metal comprises gold.

13. The polymer coating of claim 12, wherein the anodic current from CV after coating the polymer on the gold surface is higher than the current obtained from the bare gold surface.

14. The polymer coating of claim 1, wherein the coating is less than about 20 nm in thickness.

15. The polymer coating of claim 1, wherein the coating comprises a sensing probe for detecting a target, optionally wherein the sensing probe is functionalized with thiol and/or amine groups for binding to the coating.

16. The polymer coating of claim 15, wherein the sensing probe comprises a biomolecule, synthetic affinity agent, or combinations thereof.

17. The polymer coating of claim 16, wherein the biomolecule comprises a nucleic acid, an aptamer, and/or a protein.

18. The polymer of claim 15, wherein the sensing probe is multimeric.

19. The polymer coating of claim 15, wherein the target comprises a nucleic acid, a virus, a protein, or a combination thereof.

20. An electrochemical biosensor comprising the polymer coating of claim 1.

21. A device comprising the electrochemical biosensor of claim 20.

22. An assay comprising the electrochemical biosensor of claim 20 in combination with magnetic beads labelled to sandwich a target between the polymer coating and the magnetic beads.
